SEQUENCE LISTING (2 sequences)

SEQ ID NO: 1 - protein, 3144 aa, human;
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTSKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 2 - protein, 140 aa, human;
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2008/0168569 A1
(43) Pub. Date: Jul. 10, 2008

(54) Non-invasive methods and related compositions for identifying compounds that modify in vivo aggregations of disease-related polypeptides

(76) Inventors: Paul J. Muchowski, Kenmore, WA (US); John I. Clark, Seattle, WA (US)

Correspondence Address: OLYMPIC PATENT WORKS PLLC, P.O. BOX 4277, SEATTLE, WA, 98104, US

(21) Appl. No.: 10/918,708

(22) Filed: Aug. 13, 2004

(51) Int. Cl. G01N 33/00; A01K 67/00; C12N 15/63

(52) U.S. Cl. 800/3; 800/13; 435/320.1

(57) ABSTRACT

Embodiments of the present invention are directed to various high-throughput methods for identifying compounds that are useful for the treatment of various neurodegenerative diseases, including the Huntington's Disease (HD), Parkinson's Disease (PD), Alzheimer's Disease (AD), and Amyotrophic Lateral Sclerosis (ALS). Methods and compositions of the present invention enable the exogenous expression of one or more neurodegenerative-disease-related polypeptide variants within the ocular lens of an animal host. The formation of aggregates containing neurodegenerative-disease-related polypeptide variants increases the opacity of the lens, in a manner similar to the development of age-onset cataracts. The effect of a test compound in decreasing aggregate formation and/or destabilizing aggregates that contain neurodegenerative-disease-related polypeptide variants can be visually monitored and quantified in living animal hosts by employing conventional cataract-detecting instrumentation and related methods.

### TECHNICAL FIELD

The present invention relates to compositions and methods for screening compounds in order to identify active compounds that can modify the in vivo formation of aggregates containing various disease-related polypeptides and that are therefore useful for the prevention, management, and/or treatment of various neurodegenerative diseases.

### BACKGROUND OF THE INVENTION

To protect the central nervous system from noxious agents, many animals, including humans, have developed various structural barriers, such as the “blood-brain barrier” (“BBB”), so that transport of molecules into the central nervous system can be shielded by passive and active mechanisms. The transport of endogenous and exogenous molecules from the circulatory system into the brain parenchyma is regulated mainly by the BBB. The BBB forms a lipophilic membrane consisting of cerebral-capillary-endothelial cells that are tightly arranged so that intercellular junctions between cerebral-capillary-endothelial cells act to significantly reduce the permeability rate across the BBB relative to endothelial cells that compose most other capillary vessels. In addition, cerebral-capillary-endothelial cells contain a large number of efflux proteins that are effective in exporting molecules out into the lumen of a cerebral-capillary vessel. Thus, the cerebral-capillary-endothelial cells prevent the entry of various circulating toxins and pathogens from the bloodstream into the brain.

Although the blood-brain barrier provides an effective defensive mechanism, the BBB is an effective filter that inadvertently excludes most therapeutic compounds from entering into the brain and other central nervous tissues of patients. For example, for the treatment of various neurodegenerative diseases, including the Huntington's Disease (“HD”), Parkinson's Disease (“PD”), Alzheimer's Disease (“AD”), and other related diseases, therapeutic compounds need to be transported into the brain in order to interact with targets within brain cells, such as cerebral neurons. A pathological cascade involving production of self-aggregating disease-related polypeptides leads to a number of neurodegenerative diseases. Cellular components that participate in the aggregation of disease-related polypeptides are primary therapeutic targets.

FIG. 1 illustrates transport of a hypothetical drug across a blood-brain barrier for the treatment of diseases affecting the central nervous system. In FIG. 1, a hypothetical blood-brain barrier consisting of cerebral-capillary endothelial cells 102 and 103, is shown. Compounds that enter the blood stream 104 from the site of administration are transported into the capillary beds that vascularize brain tissue composed of brain cells, including neurons and glial cells, such as 110. Transport of compounds across the blood-brain barrier (BBB) can be mediated in at least two ways. For example, a lipophilic compound 106, can be transported across the plasma membranes of a cerebral-capillary-endothelial cell 102, which is again transported across the plasma membrane of a brain cell 110. In contrast, a hydrophilic compound 108, can cross the blood-brain barrier (BBB) by passing through a gap-junction space 112 between cerebral-endothelial cells 102 and 104. For effective drug transport into the tissues of the central nervous system, such as the brain, a drug must pass through the blood-brain barrier (BBB) and through the plasma membranes of individual brain cells. To ascertain the efficiency by which a drug is able to permeate through these barriers, drug bioavailability needs to be determined. However, determining the bioavailability of drugs into the brain is experimentally challenging. Furthermore, most drugs do not pass the BBB due to improper size, charge, and/or other structural properties, as described below.

Generally, for testing the bioavailability of brain-targeting drugs, active compounds are assayed individually, for example, by injecting radio-labeled compounds into living animal hosts and evaluating the effect of chemical and physical properties of each compound on the permeability rate across the BBB. The transport rate through the BBB (referred to as “BBB transport”) is inversely proportional to the molecular weight of a drug with an upper limit of approximately 600 daltons. In general, the rate of BBB transport is directly proportional to the lipophilicity of a drug. Relative to uncharged compounds, acidic compounds with ionized or charged acidic groups demonstrate reduced transfer rates, and basic compounds with ionized or charged basic groups demonstrate transfer rates comparable to neutral compounds. In addition, efflux proteins within the plasma membranes of cerebral-capillary-endothelial cells can export compounds from the cerebral endothelial cells into the luman, reducing the rate at which compounds are transported across the BBB. The characterization for the bioavailability of each active compound is very time-consuming, and the evaluation of a large number of compounds utilizing such methods is impractical.

In general, separate assays are employed for evaluating the binding activity, the bioavailability, and the therapeutic activity of a compound. Large numbers of compounds can be tested preliminarily by using an in vitro assay to evaluate the binding activity and/or reactivity of a drug with a recombinant protein target of interest. For identifying compounds for the treatment of various neurodegenerative diseases, various in vitro aggregation assays and cell-based assays that involve the over-expression of disease-related polypeptides in established cell lines can be employed. Compounds that demonstrate sufficient interaction or reactivity with a target of interest are subsequently assayed for bioavailability using animal subjects. If a test compound exhibits an acceptable bioavailability index, which can be estimated by assays described above, then the therapeutic activity of the test compound within brain cells can be determined by sacrificing diseased-animal hosts, such as disease-specific transgenic animals that over-express disease-related polypeptides within brain cells. The effect of a test compound on brain tissue can be determined by comparing histological sections removed from drug-exposed, diseased-animal hosts to histological sections of diseased-animal hosts that have not been exposed to the test compound. Histological examinations of brain sections removed from diseased-animal hosts are not only invasive, but such examinations are very labor-intensive and impractical for the evaluation of numerous compounds.

A high-throughput method for screening a large number of compounds to identify active compounds that can permeate the BBB, and that can target various neurodegenerative-disease-related proteins and protein fragments within tissues of the central nervous system is needed. Methods for efficiently evaluating large numbers of test compounds to determine the bioavailability of a compound into the various tissues of the central nervous system, including the brain, for the treatment of various neurodegenerative diseases, such as the Huntington's disease (HD), Parkinson's disease (PD), Alzheimer's disease (AD), Amyotrophic Lateral Sclerosis (ALS), and related diseases, are highly desirable.

### SUMMARY OF THE INVENTION

Embodiments of the present invention are directed to various high-throughput methods for identifying active compounds that are likely to permeate the blood-brain barrier (“BBB”), and that are useful for the treatment of various neurodegenerative diseases, including the Huntington's Disease (HD), Parkinson's Disease (PD), Alzheimer's Disease (AD), Amyotrophic Lateral Sclerosis (ALS), and related diseases. The pathogenesis of many neurodegenerative diseases leads to the aggregation of endogenous proteins, including self-aggregating polypeptide variants, that are expressed within compartments of central nervous tissues, including brain neurons. In various embodiments, methods and compositions of the present invention enable the exogenous expression of one or more neurodegenerative-disease-related polypeptide variants within the ocular lens of an animal host. The formation of aggregates containing neurodegenerative-disease-related polypeptide variants increases the opacity of the lens, in a manner similar to the development of age-onset cataracts. The effect of a test compound in decreasing aggregate formation and/or in destabilizing aggregates that contain neurodegenerative-disease-related polypeptide variants can be visually monitored and quantified in living animal hosts by employing conventional cataract-detecting instrumentation and related methods. Compounds identified by assays of the present invention that can permeate the blood-ocular barrier can be predictive of the likelihood that the identified compounds are also permeable across the BBB. Animal hosts that can express various neurodegenerative-disease-related polypeptides in both the ocular lens and the central nervous tissues are provided.

### BRIEF DESCRIPTION OF THE DRAWINGS

FIG. 1 illustrates transport of a hypothetical drug across a blood-brain barrier for the treatment of diseases affecting the central nervous system.

FIG. 2 illustrates formation of inclusion bodies containing aggregates of neurodegenerative-disease-related polypeptides.

FIG. 3 is a schematic that illustrates drug transport across a blood-brain barrier, and drug transport across a blood-ocular barrier.

FIG. 4 is a schematic of major ocular structures of a rodent eye.

FIG. 5A is a schematic of the ocular lens of a rodent eye.

FIG. 5B is a schematic of a hypothetical ocular lens containing aggregates of disease-related polypeptides within the cortex and nuclear sub-regions.

FIG. 6 is a generalized method for screening compounds to identify active compounds for the treatment of various neurodegenerative diseases, as one embodiment of the present invention.

FIG. 7A illustrates an arrangement of a cataract-detecting apparatus and digital equipment for the documentation of aggregates containing neurodegenerative polypeptides formed within the ocular lens of living animal hosts that represents one embodiment of the present invention.

FIG. 7B illustrates detection of aggregates containing neurodegenerative-disease-related polypeptides within the lens of an animal host by slip-lamp biomicroscopy that represents one embodiment of the present invention.

FIG. 7C is a digital image of aggregates containing GFP-tagged neurodegenerative-disease-related polypeptides from an anterior view of the ocular lens of a rodent that represents one embodiment of the present invention.

FIG. 7D is a digital image of aggregates containing GFP-tagged neurodegenerative-disease-related polypeptides within the ocular lens of a rodent detected by slip-lamp biomicroscopy that represents one embodiment of the present invention.

FIGS. 7E-G illustrate an exemplary set of slit-lamp images and corresponding densitometry traces.

FIGS. 7H-7I illustrate the analysis of a hypothetical densitometry trace for a compound that reduces aggregate formation in the lens of an animal host.

FIG. 8 is a schematic of an exemplary expression cassette for expressing a gene of interest within the ocular lens of an animal host, as described in Example 1.

FIG. 9 is an immunoblot analysis of lysates prepared from lens tissues of transgenic animals, as described in Example 1.

FIG. 10 is a digital image of the lens of transgenic founder mice expressing huntingtin-exon-1 variants that contain extended poly-glutamine (“Q”) domains, as described in Example 2.

FIG. 11A is a microscopic view of lens that expresses a mutant huntingtin fragment containing a non-extended poly-Q domain “25Q,” removed from a transgenic mouse at 8 weeks of age at 10× magnification, as described in Example 3.

FIG. 11B illustrates an exemplary lens section removed from a transgenic mouse that shows a thin halo (green) of inclusion bodies at 3 weeks, as described in Example 3.

FIG. 11C illustrates an exemplary lens section removed from a transgenic mouse expressing a mutant huntingtin “72Q” fragment at 8 weeks that shows a ten-fold increase in the number of inclusion bodies within the halo region (green) during this time period, as described in Example 3.

FIG. 11D illustrates an exemplary lens section of lens removed from a transgenic mouse expressing a mutant huntingtin “72Q” fragment examined for GFP fluorescence at 20× magnification, as described in Example 3.

FIG. 11E illustrates an exemplary lens section of lens removed from a transgenic mouse expressing a mutant huntingtin “72Q” fragment, incubated with an anti-GFP antibody, as described in Example 3.

FIG. 11F illustrates an exemplary lens section of lens removed from a transgenic mouse expressing a mutant huntingtin “72Q” fragment, examined for GFP fluorescence, as described in Example 3.

FIGS. 12A-C illustrate Z-series projections at 60× magnification scanned through several inclusion bodies within a lens cell, which are isolated from a transgenic mouse expressing a mutant huntingtin “72Q” fragment, as described in Example 3.

FIGS. 13A-B illustrate a cross-section of a lens fiber cell showing inclusion bodies of varying sizes (1-5 μm) formed in the lens, as described in Example 3.

FIGS. 14A-C are digital images of the lens of transgenic mice that express α-synuclein variants, as described in Example 5.

FIGS. 15A-B show digital images of microscopic sections of lens removed from a transgenic mouse expressing α-synuclean at 4 weeks of age, at 10× and 60× magnification, respectively, as described in Example 5.

FIG. 16 provides a list of various polyglutamine-neurodegenerative diseases that are targets for therapeutic intervention by compounds identified utilizing the methods of the present invention.

FIG. 17 provides a list of various amyloid diseases that involve self-aggregating polypeptides than form amyloid fibrils.

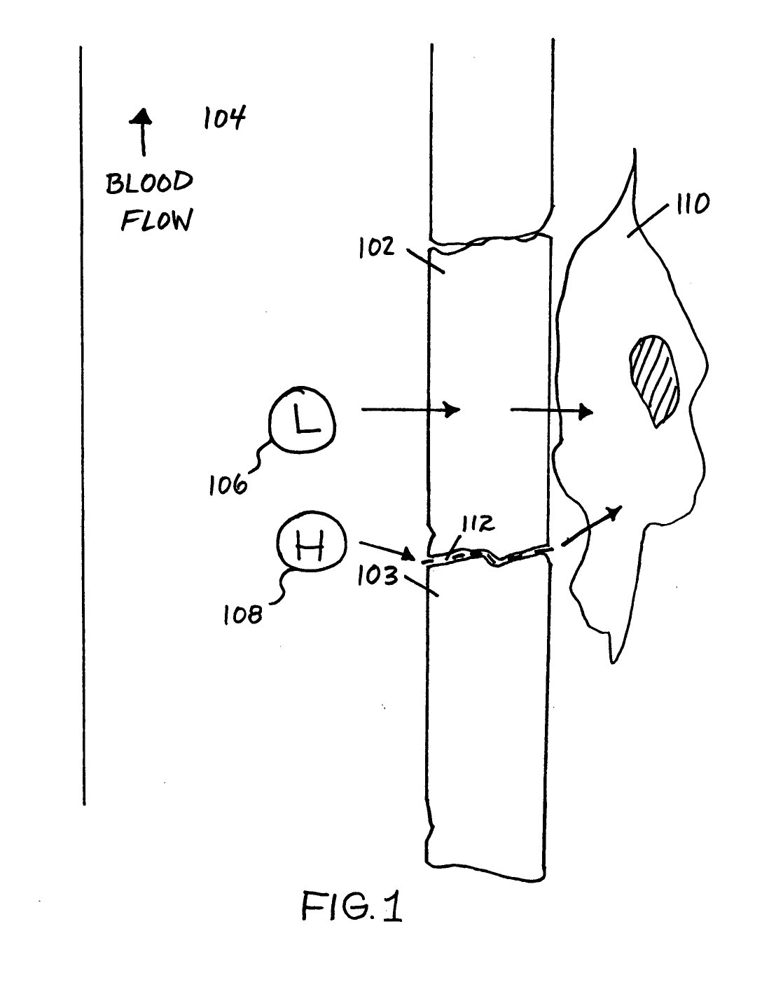

### DETAILED DESCRIPTION OF THE INVENTION

### I. Introduction

### A. Definitions

The term “neurodegenerative disease” refers to any neuropathological condition that primarily affects neurons, but excludes neoplasms, edema, hemorrhage, trauma of the nervous system, and pathologies causing neuronal death as a result of known causes such as hypoxia, poison, metabolic defects, or infections (for a comprehensive definition, refer to Przedborski et al., J. of Clinical Investigation, 111(1):3-10 (January 2003)). The term “neurodegenerative disease” includes Huntington's disease (“HD”), Parkinson's disease (“PD”), Alzheimer's disease (“AD”), Amyotrophic Lateral Sclerosis (“ALS”), and other related diseases resulting in the progressive loss of neural activity.

The terms “disease-related polypeptides,” “neurodegenerative-disease-related polypeptides,” “neurodegenerative-disease-related polypeptide variants” are used interchangeably in this disclosure to describe polypeptide molecules that can be expressed in the ocular lens of animal hosts. Neurodegenerative-disease-related polypeptides include various proteins and protein fragments that participate directly or indirectly in the pathology of various neurodegenerative diseases, such as HD, PD, AD, ALS, and related diseases. Neurodegenerative-disease-related polypeptides include full-length proteins and protein fragments that self-aggregate to form larger complexes of aggregates, or filaments, that may be referred to as either inclusion bodies when associated with HD, Lewy bodies when associated with PD, neurofilaments when associated with ALS, or amyloids when associated with AD. In addition, neurodegenerative-disease-related polypeptides include wildtype full-length proteins and protein fragments thereof that bind either directly or indirectly to self-aggregating neurodegenerative-disease-related polypeptides, and such interactions contribute to the pathology of a neurodegenerative disease. Neurodegenerative-disease-related polypeptides also include wildtype full-length proteins and protein fragments thereof that participate in the pathology of a neurodegenerative disease by means other than by interaction with self-aggregating mutant neurodegenerative-disease-related polypeptides. For example, the enzymatic activity of a neurodegenerative-disease-related polypeptide may increase the production of a deleterious self-aggregating protein at the transcriptional level, translational level, and post-translational level, which may increase the rate of aggregate formation.

The term “gene of interest” refers to a gene, or a gene fragment, that encodes “disease-related polypeptides,” “neurodegenerative-disease-related polypeptides,” and “neurodegenerative-disease-related polypeptide variants.”

The term “aggregates” refers to oligomeric complexes composed of the same or different neurodegenerative-disease-related polypeptides that localize within intracellular or extracellular compartments of diseased cells or a diseased tissue, such as the brain of diseased human patients and diseased animals afflicted with a neurodegenerative disease. In describing the present embodiments, the term “aggregates” includes fibrillar and nonfibrillar complexes composed of the same or different neurodegenerative-disease-related polypeptides that are expressed within the ocular lens of animal hosts. Aggregates can exist in any conformation, including β-pleated sheets and amyloid-like fibrils.

The term “operably-linked” refers to the joining of distinct DNA sequences to produce a functional transcriptional unit.

The term “interaction” refers to binding among discrete molecules, including polypeptides, polynucleotides, lipids, polysaccharides, and small molecules, such as compounds or drugs, in any combination. “Interaction” includes direct binding and indirect binding between one or more neurodegenerative-disease-related polypeptides of the present invention, and binding between a neurodegenerative-disease-related polypeptide of the present invention and other endogenous cellular components within tissues of an animal host, including ocular lens and brains.

The term “active compound” refers to a compound that that can interact with neurodegenerative-disease-related polypeptides, decrease the rate of aggregate formation, inhibit aggregate formation, destabilize aggregate formation, decrease the production rate of deleterious protein fragments and/or amyloidogenic polypeptides, and/or modulate the interactions between different exogenously-introduced disease-related polypeptides of the present invention that are co-expressed in the ocular lens of animal hosts. Active compounds can prevent, reduce, and/or inhibit the formation of aggregates containing neurodegenerative-disease-related polypeptide variants.

B. Co-localization of Neurodegenerative-Disease-Related Polypeptides within Aggregates of Inclusion Bodies, Lewy Bodies, Amyloids, and Neurofilaments

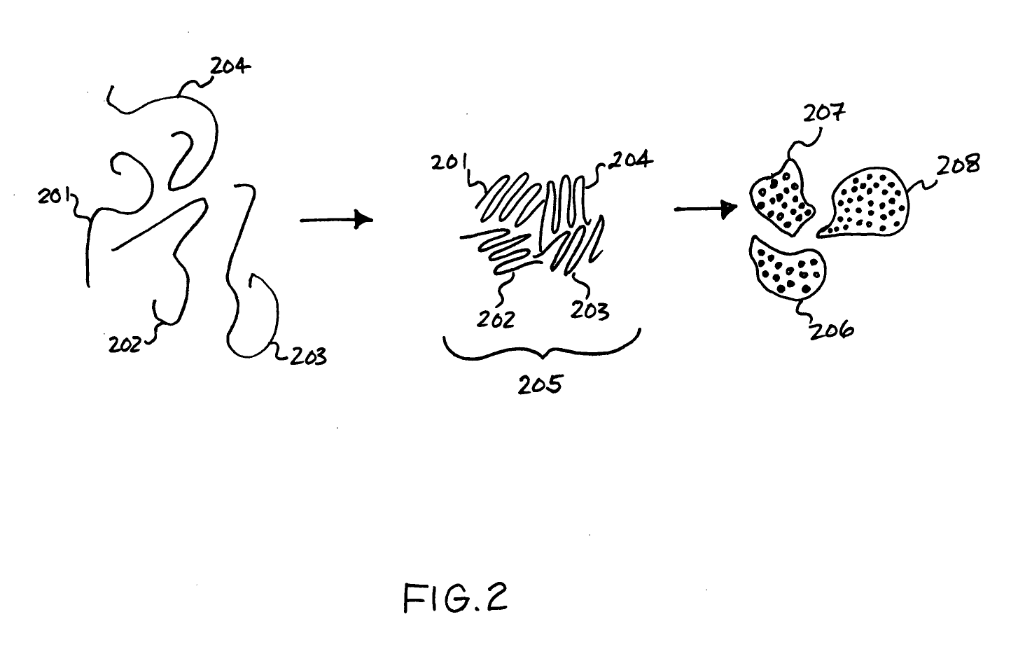

The pathogenesis of many neurodegenerative diseases, including HD, PD, AD, and ALS, involve the formation of insoluble protein aggregates and protein fibers within intracellular and/or extracellular compartments of affected cells. FIG. 2 illustrates the formation of inclusion bodies containing aggregates of neurodegenerative-disease-related polypeptides. In FIG. 2, hypothetical disease-related polypeptides, such as 201-104, are shown. Exemplary disease-related polypeptides include various self-aggregating, aberrant or mutant polypeptide variants associated with neurodegenerative diseases, such as HD, PD, AD, ALS, and related diseases. Under appropriate conditions, disease-related polypeptides, may misfold and adopt conformations, such as β-pleated sheets, and form larger aggregate complexes 205, through inter-molecular interactions. Several aggregate complexes, or aggregates, such as 206-208, may interact together to form inclusion bodies associated with HD, Lewy bodies associated with PD, amyloids associated with AD, and neurofilaments associated with ALS. Inclusion bodies and Lewy bodies can be observed in the intracellular compartments of cells, such as the nucleus or the cytoplasm, and amyloids can be formed extracellularly. For many neurodegenerative diseases, such as HD, PD, AD, and ALS, co-localization of various disease-related polypeptides with “inclusion bodies,” “Lewy bodies,” “amyloids,” and “neurofilaments” have been observed in HD patients, in PD patients, AD patients, and ALS patients, respectively. Each of these diseases are briefly described below, and various neurodegenerative-disease-related polypeptide variants that are associated with each of these diseases and related-diseases are described further in section HI, provided below.

### C. Exemplary Neurodegenerative Diseases

Huntington's Disease (“HD”) is an autosomal dominant, progressive neurodegenerative disorder caused by an expanded-CAG repeat sequence within exon 1 of a huntingtin gene, referred to as Htt or IT-15. Clinical indications of HD include progressive motor, psychiatric, and cognitive dysfunctions. A wildtype human Htt gene contains between 6-34 CAG-trinucleotide repeats within exon 1. In contrast, HD-affected persons have a mutant huntingtin gene containing, within exon 1, between 36-120 CAG repeats that include additional CAG or CAA codons that are not present in the wildtype huntingtin gene, resulting in a longer mutant huntingtin protein. Self-aggregating mutant huntingtin protein and mutant huntingtin N-terminal fragments co-localize with inclusion bodies. An “expanded-CAG-repeat sequence” of a mutant huntingtin gene encodes a domain consisting of multiple-glutamine residues (“poly-Q”). HD is one of many “polyglutamine-neurodegenerative diseases” in which a disease-associated gene contains an expanded-CAG-repeat sequence. Bennet et al., PNAS, 99(18):11634-11639 (2002) is incorporated by reference.

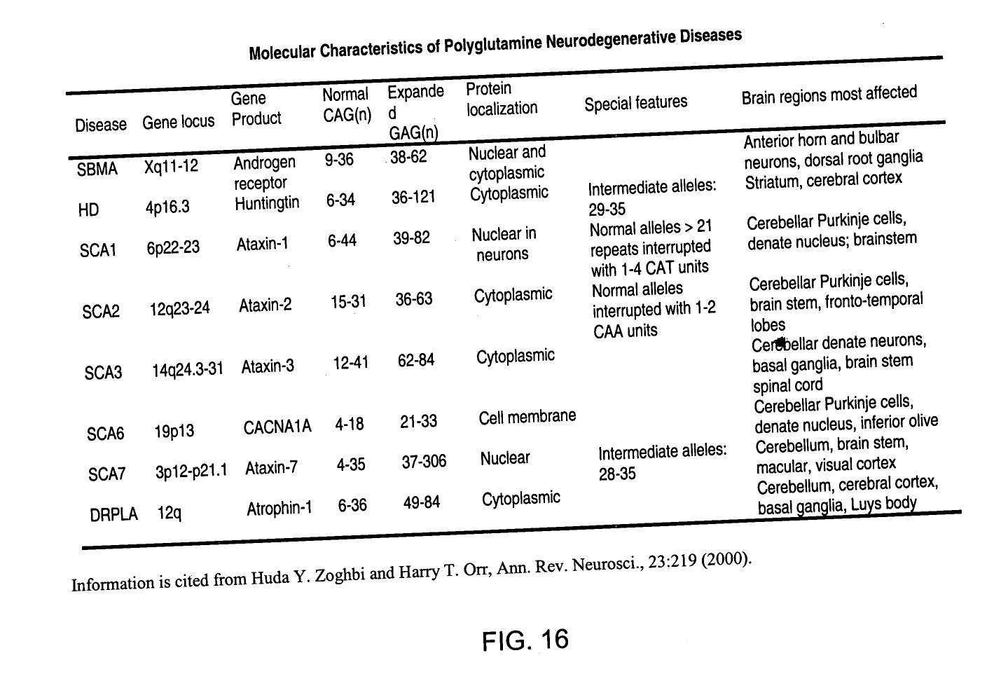

Polyglutamine-neurodegenerative diseases includes dentatorubral-pallidoluysian atrophy (“DRPLA”), spinal and bulbar muscular atrophy (“SBMA”), and various forms of spinocerebellar ataxia (“SCA”), such as SCA1, SCA2, and SCA3, which are also referred to as Machado-Joseph disease (“MJD”). FIG. 16 provides a list of various polyglutamine-neurodegenerative diseases that are targets for therapeutic intervention by compounds identified utilizing the methods of the present invention. Mutant proteins and mutant protein fragments containing an extended poly-Q domain, including ataxin-1, ataxin-2, ataxin-3, ataxin-7, atrophin 1, androgen receptor, and CACNA1A, co-localize with inclusion bodies within the cells of the central nervous system, including brain tissue, and correlate with brain lesions. For example, noticeable aggregations of disease-related polypeptides have been observed within cortical and striatal neuritis of HD-affected brains, and in some cases, these aggregations appear to correlate with substantial loss of neurons. Inclusion bodies containing self-aggregating proteins, such as ataxin-1, ataxin-2, ataxin-3, ataxin-7, atrophin 1, androgen receptor, and CACNA1A, have been observed in the nucleus, cytoplasm, dendrites, and axonal processes of diseased neurons. Aggregates that are observed in diseased neurons of affected patients and experimental animals are not observed in non-diseased patients and non-diseased experimental animals. In diseased patients and disease-specific transgenic animals that over-express disease-related polypeptides, these aggregates putatively form complexes of misfolded polypeptides that can adopt various conformations, including β-pleated sheets and fibrils. Huda Y. Zoghbi and Harry T. Orr, Annu. Rev. Neurosci., 23:217-247 (2000) is incorporated by reference.

Parkinson's Disease (“PD”) is a neurodegenerative disease that causes progressive motor, psychiatric, and cognitive dysfunctions, including symptoms such as bradykinesia, postural reflex impairment, resting tremor, and rigidity. A number of PD-related diseases include Lewy-body diseases, such as dementia of Lewy bodies (“DLB”), progressive supranuclear palsy, and multiple system atrophy (“MSA”), such as olivopontocerebellar atrophy, striatonigral degeneration, Shy-Drager syndrome, and related disorders that can result in corticobasal degeneration and frontotemporal dementia. Various conformations of wildtype and mutant α-synuclein can be detected, including: an unfolded state in solution, α-helical in the presence of lipid-containing vesicles, and β-pleaded sheet or amyloid structures in the fibrils of Lewy bodies. Diseased neural tissues frequently contain Lewy bodies, which are larger complexes of protein aggregates that contain α-synuclein as a major component. In particular, α-synuclein is a member of a small family of proteins, which are preferentially expressed in the neurons of substantia nigra. Self-aggregating wildtype α-synuclein and/or mutant α-synuclein co-localize with inclusion bodies within the cells of the central nervous system, including brain tissue, and correlate with brain lesions. Several heritable forms of PD correlate with gene products encoded by PD-associated genes, including α-synuclein, Parkin, and UCH-L1. Serpell et al., PNAS, 97(9):4897-4902 (2000) is incorporated by reference.

Alzheimer's disease (“AD”) is a progressive neurodegenerative disorder with late-onset symptoms that include dementia, loss of intellectual functions, and drastic personality changes. AD-related diseases include Creutzeldt-Jakob disease, Gerstrnann-Straussler-Scheinker syndrome, and fetal-familial insomnia. AD is one of many amyloid diseases that involve the in vivo production of filaments or fibrils, also generally referred to as amyloids, or amyloid fibrils, that contain misfolded proteins or protein fragments of a precursor protein. The predisposition to AD has been linked to mutations of several genes, such as amyloid precursor protein (“APP”), presenilin (“PS”), tau, and apolipoprotein E (“APOE”), that result in aberrant processing of amyloid precursor protein (“APP”) into self-aggregating amyloid-p-peptides. John Hardy and Dennis J. Selkoe, Science, 297:353-356, (2002) is incorporated by reference.

Amyotrophic Lateral Sclerosis (“ALS”) is a motor neuron disease involving asymmetric limb weakness and fatigue, fasciculation in the upper limbs, and/or spasticity in the legs, and degeneration of neurons of the cerebral cortex and anterior horns of the spinal cord. Relative to other neurodegenerative diseases, ALS strikes less frequently, however, ALS is the most commonly diagnosed motor neuron disease, and advances most rapidly. Indications for ALS include asymmetric limb weakness and fatigue, fasciculation in the upper limbs, and spasticity in the legs. Other disorders synonymous with ALS include Lou Gehrig's disease, Charcot disease, and motor neuron disease. ALS targets neurons of the cerebral cortex and anterior horns of the spinal cord. Approximately 5-10% of ALS cases are familial ALS (“FALS”), and a subset of FALS is attributed to substantial mutational instability within the copper/zinc superoxide dismutase type 1 (“SOD1”) gene. In diseased motor-neurons that express mutant SOD1, over-expression of endogenous wildtype SOD1 is observed. In FALS cases, SOD1 co-localizes with NF-spheroids that contain neuronal filaments (“NFs”), consisting of at least three subunits, low-molecular-weight (“LNF”), medium-molecular-weight (“MNF”), and high-molecular-weight (“HNF”) neuronal filaments. In some ALS cases, the NF-spheroids also contain intermediate filaments (“IFs”), such as α-internexin and peripherin, within proximal-axonal swellings. Ayako Okado-Matsumoto and Irwin Fridovich, PNAS, 99(13), 9010-9014 (2002) is incorporated by reference.

### II. High-Throughput Methods for Screening Compounds to Identify Candidates for the Prevention, Management, and/or Treatment of Neurodegenerative Diseases

Various embodiments of the present invention are provided below in the following sub-sections. First, a general discussion of structural features of an eye is provided to facilitate discussion of the following embodiments. Second, animal hosts that can express various neurodegenerative-disease-related polypeptides within the ocular lens of an animal host are described, including non-human transgenic animals and transgene cassettes. Third, high-throughput methods for screening compounds in order to identify active compounds that can prevent, reduce, or inhibit the formation of aggregates containing neurodegenerative-disease-related polypeptide variants are provided. Fourth, animal hosts that can express various neurodegenerative-disease-related polypeptides in both the ocular lens and the central nervous tissues are provided. Fifth, examples of various neurodegenerative-disease-related polypeptides that may be expressed in the ocular lens of animal hosts to identify active compounds for the treatment of various neurodegenerative diseases are provided.

### A. Anatomical Structures of the Eye and the Blood-Ocular Barrier (BOB)

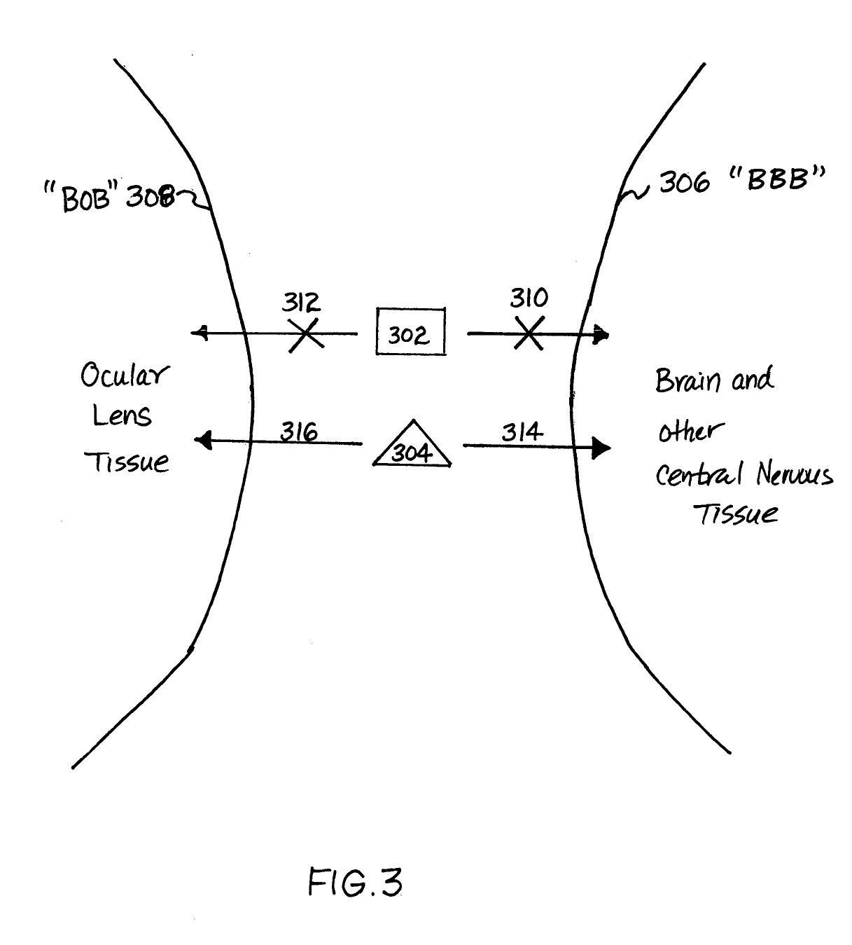

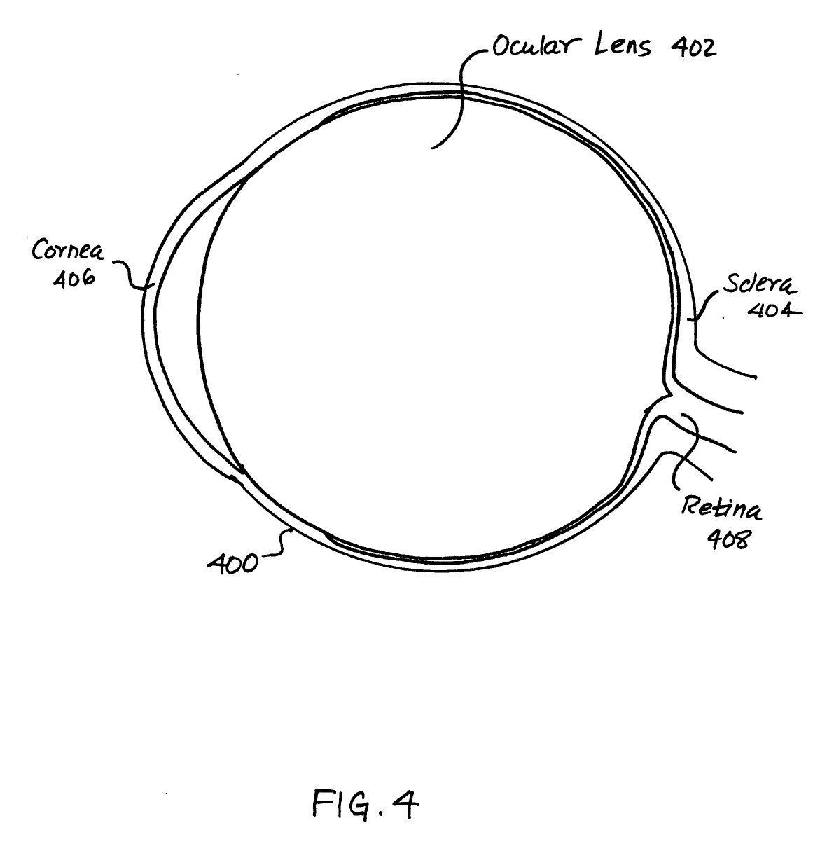

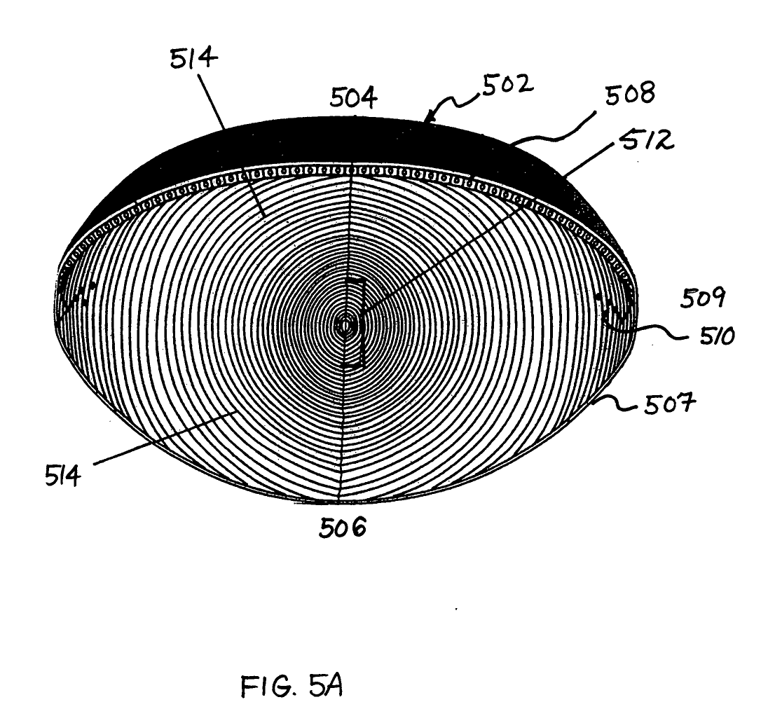

FIG. 3, FIG. 4, and FIG. 5A, described below, discuss properties shared between the BBB and the blood-ocular-barrier (“BOB”), as well as general anatomical structures of an eye, in order to facilitate the discussion of various embodiments of the present invention.

FIG. 3 is a schematic that illustrates drug transport across the blood-brain barrier, and drug transport across the blood-ocular barrier. In FIG. 3, two hypothetical compounds are shown, such as compounds 302 and 304, which can be transported by blood to various tissues of a host. Endothelial cells that compose the BBB, such as cell 306, may preclude the transport of most exogenous compounds across the BBB. Compounds, such as compound 302, that do not permeate the BBB are not accessible to brain tissue, as shown in 310. Endothelial cells that compose the BOB, such as cell 308, may preclude the transport of most exogenous compounds across the BOB. Compounds 302 that do not permeate the “blood-ocular barrier” (BOB) are not accessible to the ocular lens, as shown in 312. In contrast, some compounds, such as compound 304, can permeate the BBB, as shown in 314, and the BOB, as shown in 316. Since tight junctions are common to the BOB and the BBB, compounds that can permeate the BOB have high probability of permeating the BBB.

Vascular beds of the brain and the eye are composed of continuous capillaries that are formed by adjacent endothelial cells. Between neighboring endothelial cells that compose the BOB and BBB, narrow regions of discontinuous intercellular junctions form tiny pores having diameters of less than 1 nm. In the anterior part of the eye, a blood-aqueous barrier is formed by tight junctions between the endothelial cells of the iris capillaries and between the non-pigmented cells of the ciliary epithelium. In the posterior part of the eye, a blood-retinal barrier is formed by tight junctions between the cells of the retinal capillaries and between the cells of the retinal-pigment epithelium. Although endothelium-specific antigen (“PAL-E”) is a marker that is expressed in most endothelial cells, the PAL-E marker is not expressed within microvessels that form the BBB and the BOB. Like the BBB, the BOB is impermeable to small, water-soluable substances, such as glucose and amino acids. Like the cerebral capillaries, vital metabolic substrates are transported by carrier-mediated transport systems through the blood-aqueous barrier and the blood-retinal barrier that compose the BOB. The BBB and the BOB are functionally alike in excluding many endogenous and exogenous molecules that enter the circulatory system, including therapeutic drugs. The blood-aqueous and blood-retinal barriers prevent passage of large molecules, such as proteins, into the aqueous humor, vitreous body, and extracellular spaces of the iris and the retina. George A. Cioffi, Elisabet Granstam, and Albert Alm, Adler's Physiology of the Eye: Clinical Application, Chapter 5: The Lens, 117-147 (2003).

FIG. 4 is a schematic of major ocular structures of a rodent eye. A major portion of a rodent eye 400 is composed of an ocular lens 402 that occupies an interior region surrounded by an anterior chamber 404 and by a posterior chamber 406. The anterior chamber is covered by a cornea 408. The posterior chamber 406 occupies the space between the lens 402 and several layers of specialized epithelium, including a retina 410, a choroid 412, and a sclera 414. A cornea 406 covers the anterior surface of the eye. An optic nerve 416 converges at the posterior region of the eye. Systematic Evaluation of the Mouse Eye, Anatomy, Pathology & Biomethods. R. S Smith, editor, CRC Press, Washington D.C. (2002).

FIG. 5A is a schematic of the ocular lens of a rodent eye. A cross-section of a hypothetical ocular lens 502 is shown, having the anterior portion 404 illustrated at the top, and the posterior portion 506 illustrated at the bottom of the figure. A lens capsule 507 encloses the ocular lens. Beneath the lens capsule on the anterior side, a monolayer of epithelial cells forms an epithelium 508 that represents a region of ongoing mitosis. At the equator 509, cells differentiate into elongated fiber cells 510. As fiber cells continue to differentiate, these cells pack together tightly, and continue to move inwards towards the deep center of the spherical lens. Because fiber cells progressively internalize with the passage of time, the oldest lens cells are located at the center, and the youngest lens cells that compose the epithelium 506 are located farthest away from the center. The interior region is referred to as the nucleus 512. The outer region is referred to as the cortex 514. Generally, the ocular lens of a healthy, young animal is free of cataracts. Cataracts usually develop in the lens of aging animals when aggregates containing various crystallin proteins form within fiber cells of the nucleus 512 and the cortex 514. David C. Beebe, Adler's Physiology of the Eye: Clinical Application, Chapter 5: The Lens, 117-147 (2003).

B. High-Throughput Drug-Screening Methods to Identify Compounds that Alter Aggregate Formation

Prior to the present invention, living animal subjects have not been employed for screening a large number of compounds in order to identify compounds that can permeate the BBB, or to identify compounds that can reduce the aggregation of neurodegenerative polypeptides which usually form in the brain. In one aspect, the present invention provides compositions and methods for identifying pharmacological agents, or active compounds, that are useful for the prevention, management, and/or treatment of neurodegenerative diseases, including the Huntington's Disease (HD), Parkinson's Disease (PD), Alzheimer's Disease (AD), Amyotrophic Lateral Sclerosis (ALS), and related diseases, by employing living animal hosts. For example, compounds that can interact with disease-related polypeptides within aggregates can be used to destabilize aggregate formation, or to decrease the rate of aggregate formation so that clinical symptoms associated with these diseases can be ameliorated. In various embodiments, the present invention provides high-throughput methods for screening a large number of compounds in order to identify active compounds that can interact with neurodegenerative-disease-related polypeptides, decrease the rate of aggregate formation, destabilize aggregate formation, decrease the formation of deleterious protein fragments and amyloidogenic polypeptides, and/or modulate interactions among neurodegenerative-disease-related polypeptides of the present invention.

In the following, various embodiments of the methods of the present invention are disclosed. First, animal hosts that can be genetically engineered to express various neurodegenerative-disease-related polypeptides of the present invention within the ocular lens are described. Second, methods for expressing neurodegenerative-disease-related polypeptides within the ocular lens of animal hosts are described. Third, methods for producing neurodegenerative-disease-related polypeptides as fusion proteins that contain a fluorescent domain in order to facilitate the detection and quantification of aggregates of such disease-related polypeptides are described. Fourth, methods for detecting and measuring aggregates of neurodegenerative-disease-related polypeptides within the ocular lens of animal hosts that have been exposed to test compounds, and within the ocular lens of control animal hosts that have not be exposed to test compounds are described.

1. Expression of Neurodegenerative-Disease-Related Polypeptides within the Ocular Lens of Animal Hosts
a. An Overview

Various embodiments of the present invention provide methods for screening compounds to identify active compounds for the treatment of various neurodegenerative diseases by employing animal hosts that can be genetically engineered to exogenously express neurodegenerative-disease-related polypeptides within the ocular lens of the animal host. Aggregates of neurodegenerative-disease-related polypeptides can be formed within the ocular-lens tissue, by exogenously introducing genes that encode various neurodegenerative-disease-related polypeptides that are typically produced within the central nervous tissues of diseased patients and diseased animals. Aggregate formation in the ocular-lens tissue can convert a transparent lens to an opaque lens, similar to the development of age-onset cataracts in aging mammals. Since the effect of test compounds on brain tissue is difficult to monitor directly, the ocular-lens tissue of an animal host can be engineered to express various neurodegenerative-disease-related polypeptides that can react with test compounds. Compounds that can inhibit aggregate formation resulting from, for example, interactions between self-aggregating disease-related polypeptides, can reduce the opacity of the lens of the animal host, which can be detected by various cataract-detecting instrumentation and related methods described below. In contrast, non-active compounds that cannot reduce the opacity of the lens of the animal host will be rejected. A test compound that can reduce the lens opacity will be accepted as an active compound that can be further characterized to determine various pharmacokinetics and therapeutic activities.

FIG. 5B is a schematic of a hypothetical ocular lens containing aggregates of disease-related polypeptides within the cortex and nuclear sub-regions. A cross-section of an ocular lens, such as 520, is shown, hypothetically isolated from an animal host engineered to express neurodegenerative polypeptides within the ocular lens. Although inclusion bodies are dispersed through the depth of the lens, including the ocular nucleus and the ocular cortex, discrete inclusion bodies within one plane of the lens are shown for simplification. In one embodiment, methods of the present invention enable the exogenous production of various neurodegenerative-disease-related polypeptides within the ocular nucleus so that such aggregates of disease-related polypeptides contained within inclusion bodies or equivalent structures, such as structures 521-525, can be quantified. In another embodiment, methods of the present invention enable the exogenous production of various neurodegenerative-disease-related polypeptides within the ocular cortex so that such aggregates of disease-related polypeptides contained within inclusion bodies or equivalent structures, such as structures 526-529, can be quantified.

b. Animal Hosts

In one embodiment, the present invention is directed to animal hosts, including non-human transgenic animals, that can express, within the ocular lens, various neurodegenerative-disease-related polypeptides associated with various neuropathologies, such as HD, PD, AD, ALS, and related diseases. Such non-human transgenic animals can be utilized for screening compounds for the prevention, management, and/or treatment of HD, PD, AD, and ALS. Suitable animal hosts include any animal that can be genetically engineered to express neurodegenerative-disease-related polypeptides within the ocular lens. For example, a non-human transgenic animal, such as a rodent, can be engineered to express various neurodegenerative-disease-related polypepfides in the ocular lens. Methods for producing non-human transgenic animals are known by persons skilled in the art (Zheng et al., Molecular and Cellular Biology, 20:648-655 (1999); Arakawa et al., BMC Biotechnology, 1:7 (2001); and Hollis et al., Reproductive Biology and Endocrinology, 1:79 (2003) are incorporated by reference). Examples 1-3 describes transgenic mice that can express polypeptide variants of mutant human huntingtin, including HtEx1-25Q, HtEx1-47Q, HtEx1-72Q, and HtEx1-103Q, in the ocular lens, which were generated by the present inventors. Examples 4-5 describes transgenic mice that can express human α-synuclein variants in the ocular lens, including wildtype α-synuclein and A53T α-synuclein. Suitable examples of neurodegenerative-disease-related polypeptides include self-aggregating proteins, such as protein fragments derived from huntingtin, α-synuclein, and amyloid precursor protein (APP) as well as others.

Alternatively, genes of interest may be introduced into the ocular lens by various gene-transfer methods that permit transient gene-expression levels, and that do not require the generation of transgenic-animal hosts. For example, the present expression cassettes containing a promoter that can be activated in the lens, such as a lens-specific promoter, and a gene of interest that encodes a neurodegenerative-disease-related polypeptide may be incorporated into a recombinant, non-malignant virus particle that can infect the ocular lens, such as the Herpes Simplex Virus, cytomegalovirus (“CMV”), adenovirus and adenovirus derivative vectors, Epstein-Barr virus, and retroviruses (U.S. Pat. No. 6,204,251 is incorporated by reference). Such vector-mediated gene-expression methods enable the introduction of genes of interest into tissues of interest within animal hosts at any stage of development and growth, including adult animals. As an example, recombinant adeno-associated virus (“rAAV”) and lentivirus vectors containing α-synuclein have been used to specifically target brain neurons in primates (Kirik et al., PNAS, 100(5):2884-2889 (March 2003) is incorporated by reference). Other transient, nucleic-acid transfer methods that are emerging in the art are envisioned for introducing the expression cassettes of the present invention into the ocular lens of animal hosts without requiring the generation of transgenic hosts.

c. Expression Vectors and Expression Cassettes

An expression cassette containing a single transgene that encodes a neurodegenerative-disease-related polypeptide can be employed for pronuclei microinjections. Transgenic animals that can express two or more transgene products can be produced by crossing two independent founder lines, with each founder having a unique transgene incorporated into the genome of a founder. Alternatively, an expression cassette can be designed to express different transgenes of interest. Methods for crossing transgenic animals to produce a desired genotype is an established method known by persons skilled in the art. Exogenous genes of interest that encode neurodegenerative-disease-related polypeptides can be incorporated into expression vectors and expression cassettes. In one embodiment, various expression cassettes that enable the expression of neurodegenerative-disease-related polypeptides within the ocular lens are provided. For example, a suitable expression cassette includes a promoter element that can be activated in lens cells, such as a lens-specific promoter, and that can be operably-linked to a gene of interest, such as a transgene, that encodes a neurodegenerative-disease-related polypeptide. Optionally, a gene of interest can be operably-linked to a gene, or a gene fragment, that encodes a fluorescent polypeptide so that a fluorescently-tagged, fusion protein is produced. Suitable exogenous genes of interest that can be operably-interchanged within the expression cassettes of the present invention, and that can encode various neurodegenerative-disease-related polypeptides are further described in section III.

Examples of suitable lens-specific promoter include: mouse αA-crystallin promoter, mouse βB1-crystallin promoter, human αA-crystallin promoter, human βB1-crystallin promoter, rat αA-crystallin promoter, rat βB1-crystallin promoter, chicken βB1-crystallin, human pitx3 promoter, mouse pitx3 promoter, and rat pitx3 promoter. Generally, a βB1-crystallin promoter is stronger than a αA-crystallin promoter. Methods for constructing expression cassettes are known by persons skilled in the art. Thus, experimentations with any particular lens-specific promoter in order to optimize the expression levels for a gene of interest is within the scope of persons skilled in the art of molecular biology. In general, usage of different promoters may result in different rates of transcription. Appropriate adjustments may be needed to control the extent of lens opacification that results from the expression of a particular neurodegenerative-disease-related polypeptide of interest.

Different promoter elements, different transgenes, and different fluorophores may be interchanged within an expression cassette. Various combinations of promoters, transgenes, and fluorescent-polypeptide-encoding sequences, can be operably-linked to produce a functional expression cassette. In addition to these three elements, other DNA sequences, such as initiation signals, enhancers, insulators, termination signals, and 5′ and 3′ untranslated regions may be operably-linked to these components of an expression cassette in order to optimize gene expression in the lens. A general method for designing expression constructs that enable the expression of exogenous gene of interest, specifically in the ocular lens is disclosed in U.S. Pat. No. 5,610,294 by Lam et al., and by Tumminia et al., in Exp. Eye Res. 72:115-121 (2001), which are incorporated by reference. These authors showed that HIV-1 protease over-expression in the lens of transgenic mice, which is regulated by the αA-Cystallin promoter, produces cataract formation, whereas the inactive form of HIV-1 protease did not produce cataract formation.

d. Exemplary Fluorophores for Labeling Neurodegenerative-Disease-Related Polypeptides

Neurodegenerative-disease-related polypeptides of the present invention can be tagged by incorporating a fluorescent polypeptide. Examples of suitable fluorescent polypeptide include green fluorescent protein (“GFP”), blue fluorescent protein (“BFP”), cyan fluorescent protein (“CFP”), yellow fluorescent protein (“YFP”), red fluorescent protein (“RFP”), CGFP, enhanced yellow fluorescent protein (“EYFP), Citrine, Venus, and mutant variants of these fluorophores isolated from various organisms in the hydrozoa, cnidaria, anthozoa, and ctenophora phyla. GFP is a polypeptide of 238 amino acids that absorbs blue light with a major peak at 395 nm, and emits green light with a major peak at 509 nm. With respect to GFP from Aequorea victoria, EGFP is a GFP variant in which a serine is substituted by a threonine at position 65, and is six-fold brighter than the wildtype GFP (Clontech, Palo Alto, Calif.). BFP is a mutant variant of GFP in which a tyrosine is substituted by a histidine at position 66. In contrast to the green light of GFP, BFP polypeptide fluoresces as bright blue light. CGFP is a variant of CFP in which a threonine is substituted by a tyrosine at position 203, and has an excitation and emission wavelength that is intermediate between CFP and EGFP. EYFP is a variant of YFP in which a valine is substituted by a leucine at position 68, and a glycine is substituted by a lysine at position 69. Citrine is a variant of YFP in which a valine is substituted by a leucine at position 68, and glycine is substituted by a methione at position 69. Venus is a variant of YFP in which a phenylalanine is substituted by a leucine at position 46, a phenylalanine is substituted by a leucine at position 64, a methionine is substituted by a threonine at position 153, a valine is substituted by an alanine at position 163, and a serine is substituted by a glycine at position 175. Other polypeptides with fluorescent properties are contemplated by the present invention (Zhang et al., Nature, 3:906-918 (2002) is incorporated by reference). The neurodegenerative-disease-related polypeptides of the present invention may be tagged or labeled by alternative methods, including the use of non-GFP fluorophores.

In one embodiment, an animal host of the present invention can express a single neurodegenerative-disease-related polypeptide within the ocular lens of a host animal. Suitable examples of neurodegenerative-disease-related polypeptides include self-aggregating proteins and protein fragments derived from huntingtin, α-synuclein, and amyloid precursor protein (APP).

In another embodiment, an animal host of the present invention can express two or more different neurodegenerative-disease-related polypeptides that are each differentially-labeled with a fluorophore, such as a fluorescent polypeptide, within the ocular lens of a host animal. Suitable examples of neurodegenerative-disease-related polypeptides that can be co-expressed include a set of proteins or protein fragments that contribute to the pathogenesis of diseases by interacting together, a set of neurodegenerative-disease-related polypeptides proteins that are sequestered together within inclusion bodies or equivalent structures in order to stabilize such structures, a set of proteins that participate in the generation of misfolded self-aggregating peptides or protein fragments, and other sets that are further described in section III.

### 2. Drug-Screening Assays

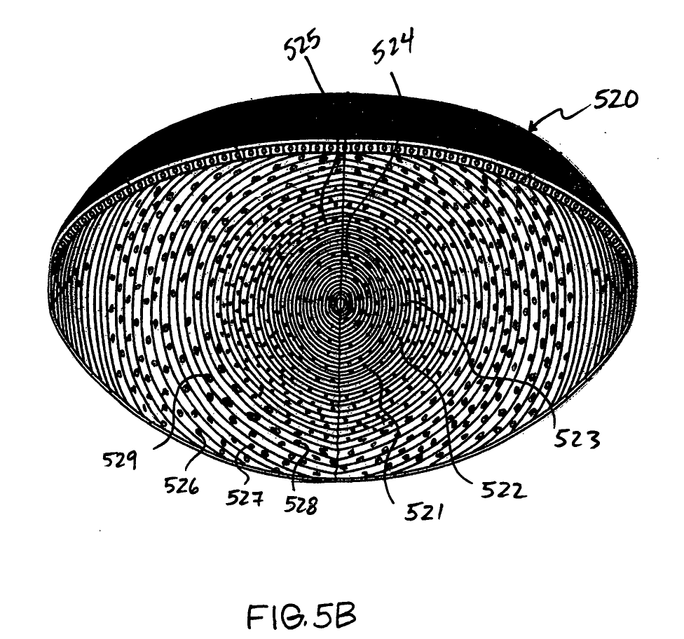

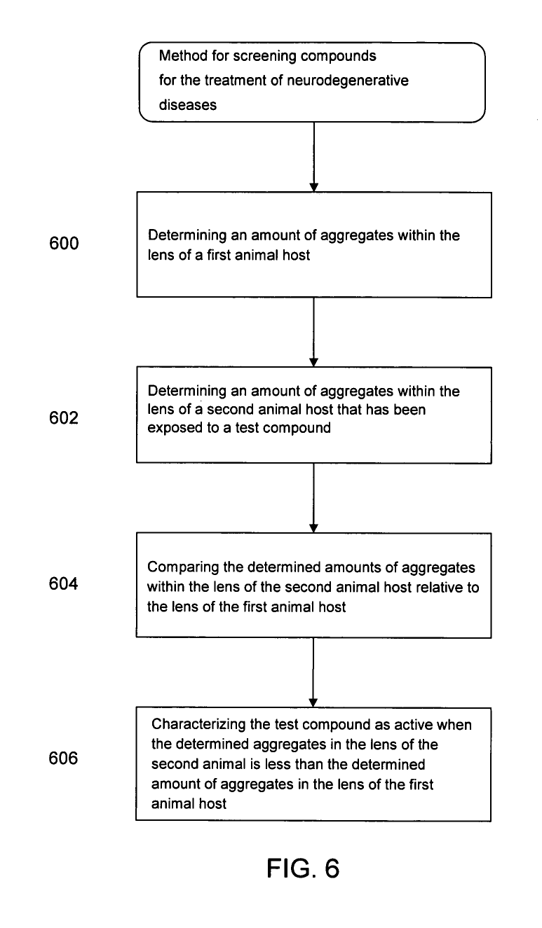

FIG. 6 is a generalized method for screening compounds to identify active compounds for the treatment of various neurodegenerative diseases that represents one embodiment of the present invention. In step 600, a first animal host that has been genetically-engineered to express one or more neurodegenerative-disease-related polypeptide within the ocular lens of the host animal, and that has not been exposed to a test compound, is evaluated to determine the amount of aggregates containing neurodegenerative-disease-related polypeptides within the lens as a reference measurement. In step 602, the amount of aggregates containing neurodegenerative-disease-related polypeptides is determined within the ocular lens of a second animal host that is genetically identical to the first animal host, and that has been exposed to a test compound. In step 604, the amount of aggregates detected within the lens of the second animal host is compared to the reference measurement. In step 606, the test compound is characterized as an active compound if the amount of aggregates detected in the lens of the second animal host is decreased from the reference measurement by a threshold decrease. In various embodiments, a decrease of at least about 10%, 15%, 20%, 25%, 30%, 35%, 40%, 45%, 50%, 55%, 60%, 65%, 70%, 75%, 80%, 85%, 90%, or 95% may be considered a threshold decrease. A decrease in an amount of aggregates includes a decrease in the size of aggregates, a decrease in the number of aggregates, and/or a decrease in the density of aggregates in the ocular lens of compound-exposed animal hosts.

In another embodiment, a reference measurement that quantifies aggregates within the lens of a statistical set of control animals that have not been exposed to any test compound can be stored in a database so that comparisons with animals of the same genetic background that have been exposed to different compounds can be made efficiently without the necessity of measuring the amount of aggregates in the lens of a first animal, or a control animal, for every compound tested.

In another embodiment, an animal host can be evaluated before the administration of a test compound and evaluated after the administration of the test compound so that the effect of the test compound on the aggregation of neurodegenerative-disease-related polypeptides can be determined. In various embodiments, an active compound decreases aggregation by a threshold decrease of at least about 10%, 15%, 20%, 25%, 30%, 35%, 40%, 45%, 50%, 55%, 60%, 65%, 70%, 75%, 80%, 85%, 90%, or 95%.

### 3. Data Analysis

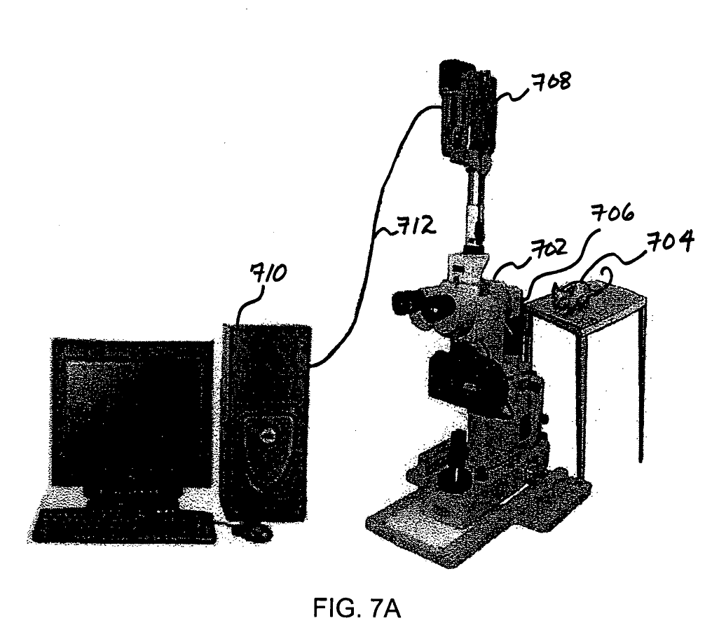

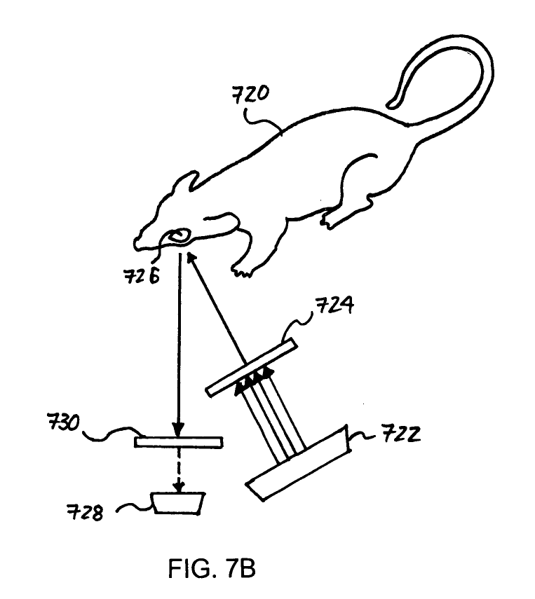

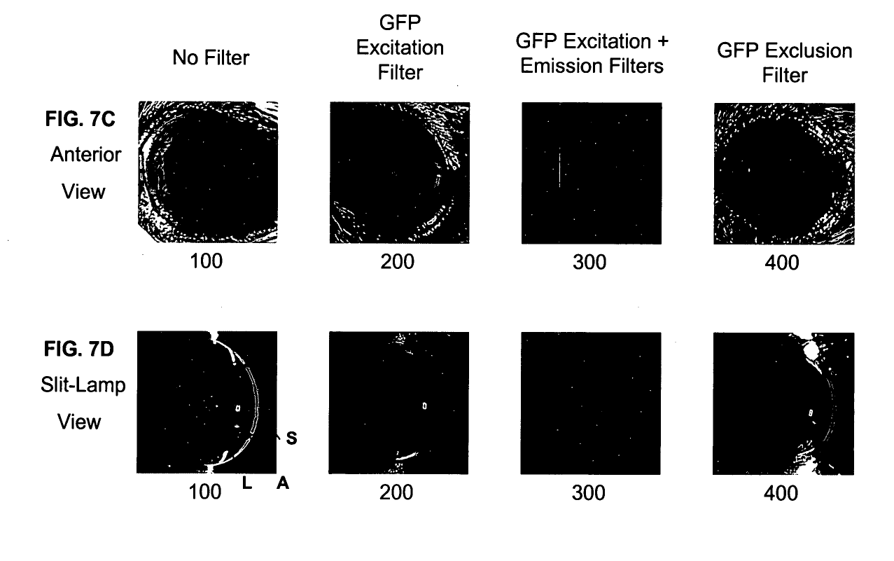

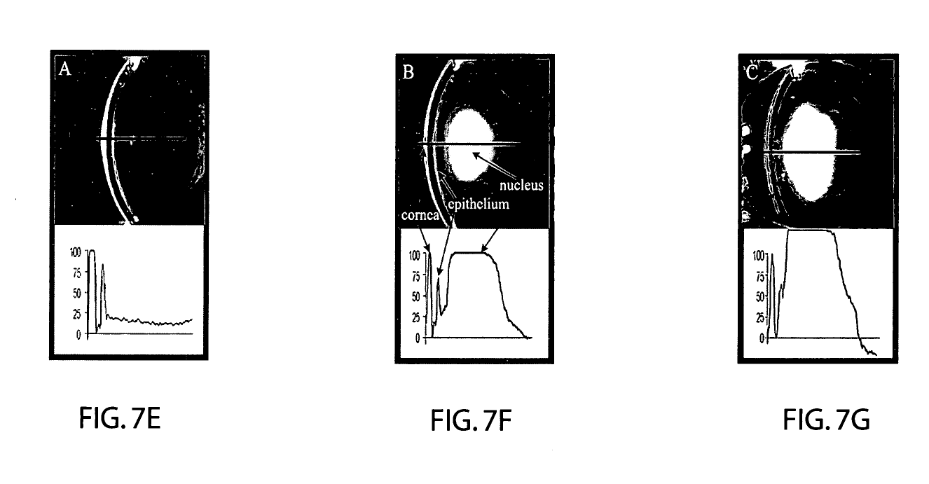

FIG. 7A illustrates an arrangement of cataract-detecting apparatus and digital equipment for the analysis and documentation of aggregates containing neurodegenerative polypeptides formed within the ocular lens of living animal hosts that represents one embodiment of the present invention. In FIG. 7A, an apparatus that can detect cataractogenesis 702 is shown. An animal host, such as a transgenic mouse 704, is placed in front of the objective lens 706 or an equivalent feature of the apparatus. Suitable cataract-detecting apparati include various types of slip-lamp biomicroscopes, laser-light spectroscopes, and modifications of these instrumentations containing composite features of both types of instrumentation. A digital-video camera 708 can be positioned above the apparatus to record the light scattering from aggregates present within the lens of an animal host observed using an apparatus, such as a slip-lamp. Video clips can be transferred from the digital-video camera to a computer 710 through a fire wire 712. Video clips can be stored in a database for automated documentation and image analysis.

Various imaging methods can be used to evaluate the ocular lens of animal hosts, including slit-lamp photography, retroillumination photography, Scheimpflug photography, video capture, and quasi-elastic (dynamic) light scattering. Digital evaluation may be preferred for evaluating animal hosts having eyes smaller than human eyes, since most imaging methods described above have been developed for human opthalmologic uses, and can be costly. In digital documentation, the location, the extent, and the intensity of lens opacity due to aggregate formation can be determined reproducibly. By rendering densitometry traces of captured digital images, which is described below in FIGS. 7E-7I, digital documentation permits a cost-effective and an objective quantification of aggregates formed within the ocular lens. Subtle changes in lens opacification that correlate with changes in aggregation resulting from an exposure to a test compound can be detected and digitally recorded to facilitate analysis and quantification.

a. Slip-Lamp Biomicroscopy for Quantifying Changes in Lens Opacity

In one embodiment, the quantification of aggregates containing neurodegenerative-disease-related polypeptides within the lens of host animals is determined by employing a slip-lamp biomicroscopy and related instrumentations that perform equivalent functions. The present inventors have developed a method for documenting lens opacification in rodents by employing slip-lamp biomicroscopy (Seeberger et al., “Digital image capture and quantification of subtle lens opacities in rodents,” Journal of Biomedical Optics, 9(1):116-120 (January/February 2004) is incorporated by reference).

FIG. 7B illustrates detection of aggregates containing neurodegenerative-disease-related polypeptides within the lens of an animal host by slip-lamp biomicroscopy that represent one embodiment of the present invention. A hypothetical animal host 720 that is genetically-engineered to express neurodegenerative-disease-related polypeptide within the ocular lens is positioned in front of a light source 722 emitted from a cataract-detecting apparatus, such as a slit-lamp. A GFP-excitation filter that transmits light of 465-498 nm wavelength known to excite GFP 724 is place between the lens 726 of the animal host and the light source 722. A photoelectric sensor 728 is placed in a suitable position to record the light scattered from aggregates within the lens of the animal host. A GFP-emission filter 730 can be positioned between the lens 726 of the animal host and the photoelectric sensor 728. Light scattered from the lens of an animal host when first exposed to a filtered light of 465-498 nm wavelength is filtered by a GFP-emission filter to transmit light having a wavelength of 515-560 nm, which appears green. Alternatively, a GFP-blocking filter can be positioned between the lens 726 of the animal host and the photoelectric sensor 728 to block the transmission of light of 475-560 nm wavelength, that includes GFP-excitation and GFP-emission ranges. A GFP-blocking filter excludes fluorescence signal originating from aggregates containing GFP-tagged neurodegenerative-disease-related polypeptides located in the ocular lens of the animal host.

FIG. 7C illustrates detection of aggregates containing GFP-tagged neurodegenerative-disease-related polypeptides from an anterior view of the ocular lens of a rodent that represent one embodiment of the present invention. Panel 100 shows a digital image of the lens of an animal host when exposed to an unfiltered light. Panel 200 shows a digital image of the lens of an animal host when exposed to a light filtered to transmit a range of 465-498 nm wavelength known to excite GFP. Panel 300 shows a digital image of light scattered from GFP-tagged aggregates located within the lens of an animal host. In Panel 300, the lens are first exposed to a filtered light of 465-498 nm wavelength by employing a GFP-excitation filter, and any background light that is not within the GFP-emission range is subsequently filtered out. The green circular image in panel 300 represents a sub-region of the lens containing aggregates of GFP-tagged neurodegenerative-disease-related polypeptides. Panel 400 shows a digital image of the lens of an animal host when the light reaching a photoelectric sensor is filtered to exclude wavelengths of GFP-excitation and GFP-emission ranges.

FIG. 7D is a digital image of aggregates containing GFP-tagged neurodegenerative-disease-related polypeptides within the ocular lens of a rodent detected by slip-lamp biomicroscopy that represent one embodiment of the present invention. A slip-lamp analysis permits the viewing of an optical cross-section of an ocular lens. Panel 100 shows a digital slip-lamp image of the lens of an animal host when exposed to an unfiltered light. In panel 100, from left to right, a spherical lens “S” is shown, which is surrounded by an anterior chamber “A,” which is covered by a cornea at the outer surface “C.” Panel 200 shows a digital slip-lamp image of the lens of an animal host when exposed to a light filtered to transmit light of 465-498 nm wavelength range known to excite GFP. Panel 300 shows a slip-lamp image of light scattered from GFP-tagged aggregates located within the lens of an animal host. In panel 300, the lens are first exposed to a filtered light of 465-498 nm wavelength by employing a GFP-excitation filter, and any background light that is not within the GFP-emission range is subsequently filtered out. The green circular image in panel 300 represents a cross-section of the lens containing aggregates of GFP-tagged neurodegenerative-disease-related polypeptides. Panel 400 shows a digital slip-lamp image of the lens of an animal host when the light reaching a photoelectric sensor is filtered to exclude wavelengths of GFP-excitation and GFP-emission ranges.

FIGS. 7E-G illustrate an exemplary set of slit-lamp images and corresponding densitometry traces. In FIGS. 7E-G, slit-lamp images that are digitally recorded (top panels) and corresponding densitometry traces (bottom panels) are shown. FIG. 7F is marked with structural features that correlate with the various slip-lamp images shown. The white central region represents the nucleus of the lens, and various anatomical structures that overlay the lens are shown as an optical cross-section. An outer-most curved white line represents scattered light incident on the cornea. To the right (or posterior) of the cornea, a darker curved line represents the aqueous chamber. Next, further to the right, a thinner white line posterior to the aqueous chamber represents the lens capsule and cells of the anterior epithelium. For each slit-lamp image, densitometry graphs are plotted along a line of pixels taken through the center of each slit-lamp image. FIG. 7E shows the slip-lamp image of a hypothetical lens without cataracts. The scattering levels of light detected within the lens is insubstantial as shown by the densitometry trace. In FIGS. 7F and 7G, the scattering levels of light detected within the lens is very substantial with the peak of the curve going off the chart.

Densitometry traces that correspond to individual slip-lamp images can be produced as follows, as one embodiment of the present invention. Digital images can be converted to gray-scale using appropriate software, such as Adobe Photoshop. A hypothetical single, horizontal line of pixels can be selected through the center of the eye and a plot profile function can be chosen. A graph of pixel brightness along all points on the selected line is produced. The pixel intensity values can be normalized to a structural feature of the eye, such as the cornea, so that comparisons can be made among different slit-lamp images. In FIGS. 7E-7G, the first peak corresponding to light scattered from the cornea is adjusted to 100% as a reference value. The minimum intensity value relative to the cornea correspond to the aqueous chamber. For example, pixel intensity values can be adjusted by the following equation: (pixel intensity value recorded−minimum of aqueous chamber)/(maximum pixel intensity value of the cornea)×100%. Adjusted pixel intensity values can be plotted as shown in FIGS. 7E-7G.

For the analysis of the lens of animal hosts that have been exposed to test compounds, two slit-lamp images, one image of lens exposed to a compound and another image of control lens, can be digitally recorded and stored so that densitometry traces may be produced. FIGS. 7H-7I illustrate the analysis of a hypothetical densitometry trace for a compound that reduces aggregate formation in the lens of an animal host. FIG. 7H shows a densitometry trace for a control lens, such as “D1” 701, that represents an intensity profile for lens that has not been exposed to any compound, where the x-axis 702 represents a scanned profile of the eye, through the center, along a horizontal line, from the cornea to the posterior region of an eye, and the y-axis 703 represents pixel-intensity values along the profile. The area under the D1 trace, such as “A1” 704, represents the total pixel-intensity within the control lens, spanning a segment of the profile shown as 713. FIG. 7I shows a densitometry trace, such as “D2” 708, that represents an intensity profile for lens that has been exposed to a test compound, where the x-axis 709 represents a scanned profile of the eye, through the center, along a horizontal line, from the cornea to the posterior region of an eye, and the y-axis 710 represents pixel-intensity values along the profile. The area under the D2 trace, such as “A2” 711, represents the total pixel-intensity within the drug-exposed lens, spanning a segment of the profile shown as 714. In FIG. 7I, the densitometry trace D1 701 represented in FIG. 7H is reproduced as a broken line, such as 712, to illustrate that the area under the D2 trace “A2” 711 that represents total pixel-intensity within the compound-exposed lens is less than the area under the D1 trace 712, or “A1” 704 of FIG. 7H. Thus, in this example, the test compound is effective in reducing the amount of light-scattering aggregates in the lens of experimental hosts. The % decrease in pixel intensity caused by the test compound can be determined, for example, by subtracting pixel intensity of A2 from pixel intensity of A1 and multiplying by 100%.

b. Quasi-elastic Laser Light Scattering Spectroscopy for Quantifying Changes in Lens Opacity

In another embodiment, the quantification of aggregates containing neurodegenerative-disease-related polypeptides within the lens of host animals is determined by employing a laser-light-scattering apparatus that provides more sensitive analysis than by using a conventional slip-lamp. The properties and advantages of quasi-elastic laser-light scattering, or dynamic light scattering, have been described in U.S. Pat. No. 5,540,226, “Apparatus for detecting Cataractogenesis;” and U.S. Pat. No. 5,392,776, “Method and apparatus for detecting cataractogenesis,” which are incorporated by reference. In principle, when aggregates of proteins within a small volume of the lens of an animal host are projected with a beam of laser light, the slowly-moving aggregates will scatter light from the lens, which can be collected as a photoelectric current that can be mathematically analyzed to obtain an autocorrelation function of the photocurrent. The autocorrelation function of the photocurrent can be used to determine the diffusivity of the scattering elements undergoing Brownian movement. The intensity of scattered light fluctuates in time because of the Brownian motion of the scattering elements composed of neurodegenerative-disease-related polypeptides. By employing a cataract-detecting instrument with laser-light-scattering features, molecular changes within aggregates of a broader size range can be quantified. Thus, smaller aggregation intermediates that form in an early stage of the aggregation process can be detected, as well as smaller aggregation intermediates that result from the destabilization of larger aggregate complexes after exposure to a compound having an aggregation-inhibition activity. Laser-light-scattering instrumentation measures autocorrelation functions for light scattered in vivo from three locations, such as the anterior, nuclear, and posterior locations, along the optic axis of the lenses of animal hosts. The presence of aggregates, the increase in the rate of aggregate formation, and the relative sizes of scattering elements composed of neurodegenerative-disease-related polypeptides, can be determined. Methods for detecting cataracts described here and other related methods are known by persons skilled in the art (M. Delaye, J. I. Clark, and G. B. Benedek, “Identification of scattering elements responsible for lens opacification in cold cataracts,” BioPhys. J., 37:647-656 (March 1982); Thurston et al., “Quasielastic light scattering study of the living human lens as a function of age,” Current Eye Research, 16:197-207 (1997); and G. B. Benedek, J. Pande, G. M. Thurston, and J. I. Clark, “Theoretical and experimental basis for the inhibition of cataract,” Progress in Retinal and Eye Research, 18(3):391-402 (1999) are incorporated by reference). A cataract-detecting apparatus containing composite features of a slit-lamp biomicroscope and laser-light-scattering spectroscopy can be used alternatively.

c. Fluorescence Microscopy

In another embodiment, the quantification of aggregates containing neurodegenerative-disease-related polypeptides within the lens of host animals is determined by employing fluorescence-microscopy techniques. A protein of interest can be tagged with GFP, a β-barrel-shaped protein that contains an amino-acid triplet (Ser-Tyr-Gly) that undergoes a chemical rearrangement to form a fluorophore, which is detectable by using an appropriate emission filter specific for the emission spectra of each variant. The resulting chimeric protein often retains the properties and function of the parent protein of interest when expressed in cells, and therefore can be used as a fluorescent reporter to study protein-protein interactions. Advances in molecular engineering of the GFP variants, have resulted in optimized expression of GFP in different cells types, and generation of GFP variants with more favorable spectral properties, including increased brightness, increased stability in broader pH ranges, and increased photo stability.

In vivo interaction between differentially-labeled neurodegenerative-disease-related polypeptides of the present invention, can be determined by fluorescence-based techniques, such as fluorescence resonance energy transfer (“FRET”) and fluorescence correlation spectroscopy (“FCS”), using a conventional fluorescence microscope. In FRET, distinct proteins of interest are labeled differentially, with one protein of interest acting as a donor, and the other protein of interest acting as an acceptor. A FRET pair, such as CYAN fluorescent protein (CFP) and yellow fluorescent protein (YFP), is chosen so that the emission spectrum of the donor, such as CFP significantly overlaps with the excitation spectrum of the acceptor, such as YFP. When CFP and YFP are in very close proximity to one another, certain parts of the fluorophores can undergo a process known as FRET, in which the CFP excitation results in sensitized fluorescence emission by YFP, when energy absorbed by CFP is transferred to YFP. CFP fluorescence is concomitantly quenched. Suitable FRET pairs include GFP/DsRed, CY3/CY5, GFP/CY3, and GFP/CY5 for the quantification of interactions occurring among neurodegenerative-disease-related polypeptides of the present invention. Methods for FRET microscopy and related data analysis are known by persons skilled in the art (Lippincott-Schwartz et al., Nature, 2:444-456 (2001); and Kim et al., Nature Cell Biology, 4:826-831 (2002) are incorporated by reference).

FRET is strongly dependent on the distance between a donor and an acceptor, so that the emission spectrum of an acceptor is observed only when the donor and acceptor are in close proximity. FRET microscopy complements fluorescence co-localization studies by providing a read-out of the molecular proximity between the donor and acceptor proteins. The amount of energy transferred at a given separation distance is dependent on a particular donor/acceptor pair. To optimally detect and quantify protein-protein interactions using FRET microscopy, several FRET pairs and labeling schemes should be tested. A donor fluorophore, such as CFP, and an acceptor fluorophore, such as YFP, should not come closer than approximately 30 Angstrom from one another. FRET microscopy is not limited to the use of GFP-chimeric proteins. In fact, samples for FRET measurements can be prepared by immunofluorescence techniques, or by using fluorescently-tagged proteins.

Fluorescence correlation spectroscopy (“FCS”) detects either changes in the diffusion or the co-diffusion of bound species. FCS measurements of fluorescently-labeled proteins can be determined using a conventional confocal microscope at any location within a cell. FCS measures the fluctuations in the photons resulting from fluorescently-labeled molecules diffusing in and out of a defined volume. These fluctuations reflect the average number of fluorescently-labeled molecules in a volume, and the characteristic time of diffusion for each molecule across the confocal volume, which can be converted to diffusion constants. FCS is highly sensitive to extremely low concentrations of fluorophores, and is sensitive to the photophysical properties of fluorophores. FCS can also readily detect several diffusing species, and therefore can be used as a sensitive probe of protein-protein interactions.

Sensitive techniques that enable the detection of smaller aggregate intermediates that are formed in non-adult transgenic animals may permit the use of younger non-adult transgenic animals for compound screening. More importantly, subtle changes in aggregation states that are induced by a drug candidate can be detected more reliably by employing increasingly sensitive techniques, which includes techniques that are disclosed as well as other emerging techniques. Furthermore, increasingly sensitive techniques will decrease the total length of time needed to evaluate each individual transgenic animal exposed to a test compound so that a larger set of animal hosts and correspondingly a larger set of drug candidates may be evaluated in any given time, thereby increasing the efficiency of analysis.

### C. Methods for Pre-screening Compounds to Identify Candidates for Blood-Brain Barrier Transport

In one embodiment, compounds identified by the methods of the present invention that can permeate the blood-ocular barrier have a significant likelihood that the identified compounds can also permeate the blood-brain barrier based on similar properties. Compounds that can permeate the blood-ocular barrier may be identified in several ways. For example, compounds that are determined to be active compounds by causing the destabilization of aggregates or the reduction in aggregate formation are those that can permeate the blood-ocular barrier. In addition, compounds that are labeled, for example, by incorporating a fluorophore, can be administered systemically to an animal host. The lens of the animal host exposed to a test compound can be monitored for the presence of fluorescently-labeled compound by using a fluorescence microscope. Detection of a fluorescently-labeled compound in the lens of the animal host indicates that the fluorescently-labeled compound of interest can permeate the blood-ocular barrier. Such compounds are also likely candidates for therapeutics that need to target central nervous tissues, including brain cells.

### D. Methods for Identifying Compounds Having Therapeutic Activity in the Central Nervous Tissue

Various embodiments of the present invention provide methods and related compositions for identifying compounds that demonstrate therapeutic activity in a central nervous tissue, including brain cells. An animal host that can express neurodegenerative-disease-related polypeptides in both the ocular lens and the central nervous tissues of the animal host can be employed for determining at least: (1) whether a test compound can decrease aggregates within the lens of a host animal; (2) whether a test compound can permeate a BBB; and (3) whether a test compound can decrease aggregates within the central nervous tissues of a host animal. For example, an animal host that can express neurodegenerative-disease-related polypeptides in both the ocular lens and in the central nervous tissues, such as a brain, can be systemically administered with a test compound. The lens of the animal host can be monitored to determine whether the test compound can permeate a blood-ocular barrier and decrease aggregates within the lens of the animal host. If so, then the animal host may be sacrificed to determine whether the test compound has an effect on the aggregation of neurodegenerative-disease-related polypeptides in the central nervous tissues, such as the brain. Histological examinations of the central nervous tissues of the animal host exposed to the test compound can be determined and compared with the histological examinations of the central nervous tissues of a control animal host that is genetically identical to the compound-exposed animal host, and that has not been exposed to the test compound. A decrease in an amount of aggregates includes a decrease in the size of aggregates, a decrease in the number of aggregates, and/or a decrease in the density of aggregates in the central nervous tissue of compound-exposed animal host. Such decrease in aggregation within central nervous tissue, such as a brain, indicates that the compound of interest has therapeutic activity within central nervous tissues.

Animal hosts that can express neurodegenerative-disease-related polypeptides in both the ocular lens and in the central nervous tissues can be produced in various ways. For example, suitable animal hosts include any animal that can be genetically engineered to express neurodegenerative-disease-related polypeptides within the ocular lens and the central nervous tissue, including brain tissue. For example, a non-human transgenic animal, such as a rodent, can be engineered to express various neurodegenerative-disease-related polypeptides in the ocular lens and in the brain of an animal host. Methods for producing non-human transgenic animals are known by persons skilled in the art (Zheng et al., Molecular and Cellular Biology, 20:648-655 (1999); Arakawa et al., BMC Biotechnology, 1:7 (2001); and Hollis et al., Reproductive Biology and Endocrinology, 1:79 (2003) are incorporated by reference). For example, a first founder transgenic animal that can express one or more neurodegenerative-disease-related polypeptides in the ocular lens can be crossed with a second founder transgenic animal that can express one or more neurodegenerative-disease-related polypeptides in the central nervous tissue to produce a suitable animal host. Examples of non-human transgenic animals that can express one or more neurodegenerative-disease-related polypeptides in the central nervous tissue and methods for producing such transgenic animals are disclosed in issued patents, such as Hayden et al., U.S. Pat. No. 5,849,995; St. George-Hyslop et al., U.S. Pat. No. 5,986,054; and Games et al., U.S. Pat. No. 6,717,031. Generally, an expression cassette that enables the expression of neurodegenerative-disease-related polypeptides within the central nervous tissue, such as brain tissue, includes a promoter element that can be activated in central nervous tissues, such as a promoter that is activated in brain cells, and that can be operably-liked to a gene of interest, such as a transgene, that encodes a neurodegenerative-disease-related polypeptide. Suitable exogenous genes of interest that can be operably-interchanged within the expression cassettes of the present invention, and that can encode various neurodegenerative-disease-related polypeptides are further described in section III.

Alternatively, genes of interest may be introduced into a central nervous tissue by various gene-transfer methods that permit transient gene-expression levels, and that do not require the generation of transgenic-animal hosts. For example, the present expression cassettes containing a promoter that can be activated in a central nervous tissue and a gene of interest that encodes a neurodegenerative-disease-related polypeptide may be incorporated into a recombinant, non-malignant virus particle that can infect the central nervous tissue. Such vector-mediated gene-expression methods enable the introduction of genes of interest into tissues of interest within animal hosts at any stage of development and growth, including adult animals. For example, recombinant adeno-associated virus (“rAAV”) and lentivirus vectors containing α-synuclein have been used to specifically target brain neurons in primates (Kirik et al., PNAS, 100(5):2884-2889 (March 2003) is incorporated by reference). Other transient, nucleic-acid transfer methods that are emerging in the art are envisioned for introducing the expression cassettes of the present invention into the central nervous tissues of animal hosts without requiring the generation of transgenic hosts.

### E. Administration of Pharmaceutical Compositions

Suitable compounds may be derived from various chemical libraries of small molecule compounds and biological mixtures, such as fungal, bacterial, and algal extracts known by persons skilled in the art. Pharmaceutical formulations for effective delivery of pharmaceutical compounds of the present invention will vary depending on the pharmaceutical compound of interest and mode of administration. Suitable pharmaceutical carriers, formulations, and administration techniques described here are commonly known by persons skilled in the art (Remington's Pharmaceutical Sciences, Meade Publishing Co., Easton, Pa. (1989), which is incorporated in its entirety). Pharmaceutical compounds can be administered by various methods, including by injection, oral administration, inhalation, transdermal application, or rectal administration. For oral administration, suitable formulations containing a pharmaceutical compound and pharmaceutically-compatible carriers can be delivered in various forms, such as tablets or capsules, liquid solutions, suspensions, emulsions, and the like. For inhalation, suitable formulations containing a pharmaceutical compound and pharmaceutically-compatible carriers can be delivered as aerosol formulations that can be placed into pressurized propellants, such as a dichlorodifluoromethane, propane, nitrogen, and the like. In order to transport compounds of interest into the circulation of an animal host, pharmaceutical compositions containing compounds and physiologically-acceptable carriers may be systemically-administered to an animal host.

As one embodiment, a pharmaceutical composition containing a test compound of interest and a physiologically-compatible carrier is administrated systemically to an animal host of the present invention that can express one or more neurodegenerative-disease-related polypeptide within the ocular lens. Suitable systemic administration methods include intramuscular, intravenous, intraperitoneal, and subcutaneous injections. Pharmaceutical compositions can be formulated in liquid solutions, preferably in physiologically-compatible buffers, such as Hank's solution or Ringer's solution. Alternatively, slow-acting compounds may be formulated with suitable polymeric or hydrophobic materials, which may be implanted subcutaneously or intramuscularly.

As another embodiment, a pharmaceutical composition containing a test compound of interest and a physiologically-compatible carrier is administrated systemically to an animal host of the present invention by oral administration, transmucosal administration, and transdermal administration. For example, for compounds that cannot be delivered by systemic injections, test compounds may be incorporated into gels or ointments that are administered transdermally. Transmucosal administration may be achieved using nasal sprays or using suppositories. Oral administration may be achieved by adding compounds to a drink or a food supply.

In another embodiment, a pharmaceutical composition can be administrated by intraocular application. Pharmaceutical compounds and pharmaceutically-compatible carriers can be formulated as an opthalmological formulation in the form of eye drops or an equivalent, which can be introduced into one or more chambers of the eye, such as the anterior chamber, or near the eye. Such formulations cannot enter the lens directly through an anterior chamber or an anterior epithelium when applied onto the cornea of the eye, but compounds will be drained from the eyes, transported by blood to a brain, and transported across a blood-ocular barrier (BOB) into the ocular lens.

In various embodiments, the methods of the present invention can be used to screen dozens of compounds, hundreds of compounds, and thousands of compounds. As one embodiment, the present methods and related compositions can be used for predicting the bioavailability of a compound into the brain and other tissues of the central nervous system. Suitable compounds include compounds that have demonstrated sufficient in vitro target specificity by an in vitro assay, and the bioavailability needs to be determined in a living animal subject. Examples of an in vitro assay to initially determine in vitro target specificity is described in Houseman et al., U.S. Pat. No. 6,420,122.

As another embodiment, the present methods and related compositions can be used initially to screen naïve compounds that have not been pre-screened by an in vitro target specificity assay. In this embodiment, the in vitro target specificity assay need not be performed since target specificity can be tested in vivo by introducing test compounds into the circulation of a living animal host, and assaying whether the test compound affects the aggregation of neurodegenerative-disease-related polypeptides. If a change in the amount of aggregates is detected in the lens when the animal host is exposed to a test compound, then the test compound demonstrates in vivo target specificity. In addition, a change in the opacity of the lens indicates that the test compound is able to permeate the blood-ocular barrier, and that the test compound is likely to permeate a blood-brain barrier. Furthermore, various parameters may be assayed by the methods of the present invention, including determining the cumulative effect of a test compound, testing various drug combinations, testing various drug dosages, testing various schedules, and any combinations of these and other parameters.

### III. Ocular-Lens Expression of Neurodegenerative-Disease-Related Polypeptides

Examples of self-aggregating neurodegenerative-disease-related polypeptides with corresponding NCBI accession numbers (“gi”) include: huntingtin (39777597), atrophin-1 (4503397), androgen receptor (105325), ataxin-1 (1710863), ataxin-2 (1679684), ataxin-3 (4530184), CACNA1A (3873285), ataxin-7 (3192948), α-synuclein (586067), amyloid precursor protein (APP) (609449), tau (602471), β-amyloid peptide (435822), low-molecular-weight neuronal filament (LNF) (5453762), α-internexin (13623507), and peripherin (501830).

Examples of neurodegenerative-disease-related polypeptides with corresponding NCBI accession numbers (“gi”) include: N-Cor (3510603), mSin3a(726286), CBP (c-AMP-responsive-element-binding protein) (4321116), α-adaptin (4314340), α1-antichymotrypsin (177809), synphilin-1(4836161), parkin (3063388), UCH-L1 (21361091), hip-1 (38045919), caspase-1 (2642241), caspase-2 (30583319), caspase-3 (16516817), caspase-6 (29124999), caspase-8 (4583149), calpain (689776), aspartyl protease (5668578), histone deacetylase 2 (HDAC2) (3023939), transglutaminases (107479), polyglutamine-binding-protein-1 (PQBP1) (5031957), β-synuclein (12804099), γ-synuclein (4507113), SOD1 (680522), apolipoprotein E (APOE) (4105704), hip-1 (30410777), presenilin PS-1(1589007), and presenilin PS-2(4432960).

Two sequence identifiers are listed. Human huntingtin designated as SEQ ID NO: 1 exemplifies a wildtype reference sequence. Human α-synuclein (also referred to as “NACP”) designated as SEQ ID NO:2 exemplifies a wildtype reference sequence.

### A. Exemplary Targets for the Therapy of HD

As a disease model for HD, several transgenic mice that can express mutant huntingtin protein and mutant huntingtin fragments, containing different lengths of an poly-Q domain encoded by an extended-CAG repeat, within brain neurons, have been developed (U.S. Pat. No. 5,849,995; Cha et al., Proc. Natl. Acad. Sci. USA, 95:6480-6485 (May 1998)). Inclusion bodies containing aggregates of mutant variants of a huntingtin fragment observed in HD-affected patients can be detected in the brain neurons of HD-specific transgenic mice. Exogenous expression of mutant N-terminal huntingtin fragments containing an extended poly-Q tract is sufficient for causing HD-like phenotypes in transgenic mice, including behavioral symptoms, such as an irregular gait, abrupt shuddering movements, resting tremor, and epileptic seizures.

In one embodiment, the present methods and compositions can be used to identify compounds that can inhibit the self-aggregation of mutant huntingtin, or mutant huntingtin fragments, for the prevention, management, and/or treatment of HD and related diseases. Neurodegenerative-disease-related polypeptides of the present invention that can be expressed in the lens of animal hosts include mutant variants of huntingtin and fragments thereof that are encoded by various mutant huntingtin genes containing an expanded-CAG-repeat sequence, such as a mutant human huntingtin, a mutant human huntingtin fragment, a mutant mouse huntingtin, a mutant mouse huntingtin fragment, a mutant rat huntingtin, mutant rat huntingtin fragment, and others. Mutant mammalian huntingtin genes can be isolated from naturally-occurring populations, or can be generated in a laboratory by various mutagenesis methods known to persons skilled in the art. An “expanded-CAG-repeat sequence” is a sub-sequence of a mutant huntingtin gene that contains more CAG and/or CAA codons than the total number of CAG/CAA codons within a non-extended CAG repeat sequence of a wildtype huntingtin gene. For example, a pre-existing CAG-repeat sequence within a known human wildtype-huntingtin gene, such as SEQ ID NO:1, can be extended by molecular genetic techniques to generate various mutant huntingtin genes containing “an extended-CAG-repeat sequence” that comprises more than 35 CAG codons, 35 CAA condons, or a mixture of CAG and CAA codons that exceeds 35 in total. Animal hosts that can express mutant mammalian huntingtin protein variants encoded by mutant huntingtin genes, including gene of humans and rodents, within the ocular lens can be used to screen for compounds that can inhibit self-aggregating interactions between mutant huntingtin variants, and that can inhibit interactions between mutant huntingtin variants and other cellular proteins that participate in HD pathogenesis.

In another embodiment, the present methods and compositions can be used to identify compounds that can inhibit the proteolytic activity of proteases that generate N-terminal huntingtin fragments for the prevention, management, and/or treatment of HD and related diseases. These N-terminal huntingtin fragments can form protein aggregates in the nucleus, cytoplasm, processes of neurons in humans and in animals, and cellular systems. For example, soluble mutant huntingtin fragments in the cytoplasm can engage in pathological interactions with various proteins in the mitochondria. Aggregates of huntingtin fragments can sequester other cellular proteins including chaperones, proteasomal proteins, normal huntingtin, and various transcription factors. Suitable neurodegenerative-disease-related polypeptides of the present invention that can be expressed in the ocular lens of animal hosts include proteases that can cleave full-length mutant huntingtin into deleterious N-terminal huntingtin fragments, such as caspase-2, caspase-3, caspase-6, calpain, and aspartyl proteases. N-terminal huntingtin fragments generated by caspases have been identified in the brains of HD patients. Huntingtin can be cleaved in vitro at amino acids 513 and 552 by caspase-3, at amino acid 586 by caspase-6, and at amino acid 552 by caspase-2, calpain, a calcium-dependent protease, may be involved in huntingtin proteolysis. The activities of active and inactive calpains are increased in the brains of HD patients, calpain co-localizes with huntingtin aggregates, and huntingtin cleavage products are similar in size to those generated by recombinant calpains. Animal hosts that can express proteases that can cleave full-length mutant huntingtin, such as caspase-2, caspase-3, caspase-6, calpain, and aspartyl proteases, within the ocular lens can be used to screen for compounds that can inhibit the generation of N-terminal huntingtin fragments. M. Flint Beal and Robert J. Ferrante, Nature Reviews, Neuroscience, 5: 373-384 (May 2004) is incorporated by reference.

In another embodiment, the present methods and compositions can be used to identify compounds that can inhibit the activity of proteins that regulate apoptosis, or programmed cell death, for the prevention, management, and/or treatment of HD. Apoptotic-regulatory pathways can be activated by the onset of a number of neurodegenerative diseases, including Huntington's disease. Increased levels of neuronal cytochrome c, caspase-1, caspase-3, and caspase-8 have been observed in post-mortem brain tissue of HD patients. Animal hosts that can express proteins that regulate apoptosis in brain neurons, such as caspase-1, caspase-3, caspase-8, and neuronal cytochrome c within the ocular lens can be used to screen for compounds that inhibit apoptosis. M. Flint Beal and Robert J. Ferrante, supra.

In another embodiment, the present methods and compositions can be used to identify compounds that can inhibit interactions between a transcription factor and a mutant huntingtin protein or a fragment thereof for the prevention, management, and/or treatment of HD. For example, the CREB-binding protein (“CBP”) is recruited into aggregates of polyglutamine-containing proteins in mammalian cells. CBP, which localizes to the nuclear compartment of a cell can be removed from the nucleus and sequestered within cytoplasmic aggregates containing huntingtin. In addition, interactions between CBP and huntingtin preclude the histone-acetyl-transferase activity of CBP which leads to decreased global gene transcription levels. Furthermore, N-terminal huntingtin fragments can interact with the transcription factor “SP1,” resulting in the suppression of SP1-transcription activity in cultured cells. A reduction in SP1-binding activity to a promoter for dopamine B2 receptors has been observed in the post-mortem brain tissue of HD patients. Other transcription factors that can bind huntingtin include the nuclear-receptor-co-repressor 1 (“Ncor1”) and the TATA-binding protein (“TBP”). Animal hosts that can co-express a transcription factor, such as CBP, SP1, Ncor1, and TBP, in addition to a mutant huntingtin variant within the ocular lens can be used to screen for compounds that can inhibit interactions between a transcription factor and mutant huntingtin variants. M. Flint Beal and Robert J. Ferrante, supra.

In another embodiment, the present methods and compositions can be used to screen compounds that inhibit the activity of histone deacetylases (“HDAC”) for the prevention, management, and/or treatment of HD. Inhibition of HDAC increases gene transcription, increases SP1 acetylation, and inhibits oxidative neuronal death. Animal hosts that can express histone deacetylases (HDAC) within the ocular lens can be used to screen for compounds that can inhibit the activity of histone deacetylases (HDAC). M. Flint Beal and Robert J. Ferrante, supra.

In another embodiment, the present methods and compositions can be used to screen compounds that inhibit the activity of transglutaminases for the prevention, management, and/or treatment of HD. In general, transglutaminases catalyze the formation of crosslinkages between glutamine and lysine residues in proteins. An increase in transglutaminase activity in the post-mortem brain tissue of HD patients correlates with pathological severity, and correlates with increased levels of y-glutamyl-lysine in cerebrospinal fluid and in aggregates containing huntingtin. The resulting y-glutamyl-lysine covalent bonds are resistant to proteolysis. Animal hosts that can co-express transglutaminases and mutant huntingtin variants within the ocular lens can be used to screen for compounds that can inhibit the activity of transglutaminases. M. Flint Beal and Robert J. Ferrante, supra.

### B. Exemplary Targets for the Therapy of Polyglutamine-Neurodegenerative Diseases

In one embodiment, the present methods and compositions can be used to identify compounds that can inhibit the self-aggregation of various mutant polyglutamine-containing proteins and fragments thereof for the prevention, management, and/or treatment of polyglutamine-neurodegenerative diseases listed in FIG. 16. Neurodegenerative-disease-related polypeptides of the present invention that can be expressed in the lens of animal hosts include mutant variants of ataxin-1, ataxin-2, ataxin-3, ataxin-7, atrophin 1, androgen receptor, and CACNA1A. Mutant mammalian genes of ataxin-1, ataxin-2, ataxin-3, ataxin-7, atrophin 1, androgen receptor, and CACNA1A can be isolated from naturally-occurring populations, or can be generated in a laboratory by various mutagenesis methods known to persons skilled in the art. An “expanded-CAG-repeat sequence” is a sub-sequence of a mutant variant of ataxin-1, ataxin-2, ataxin-3, ataxin-7, atrophin 1, androgen receptor, and CACNA 1A that contains more CAG and/or CAA codons than the total number of CAG/CAA codons contained within a non-extended-CAG-repeat sequence of a corresponding wildtype gene. Animal hosts that can express mutant mammalian polypeptide variants of ataxin-1, ataxin-2, ataxin-3, ataxin-7, atrophin 1, androgen receptor, and CACNA1A encoded by the respective mutant genes, including genes of humans and rodents, within the ocular lens can be used to screen for compounds that can inhibit self-aggregating interactions between such mutant variants, and that can inhibit interactions between such mutant variants and other cellular proteins that participate in the pathogenesis of polyglutamine-neurodegenerative diseases listed in FIG. 16.

In another embodiment, the present methods and compositions can be used to identify compounds that can inhibit interactions between a transcription factor and various mutant polyglutamine-containing proteins and fragments thereof, which includes ataxin-1, ataxin-2, ataxin-3, ataxin-7, atrophin 1, androgen receptor, and CACNA1A, for the prevention, management, and/or treatment of various polyglutamine-neurodegenerative diseases. For example, the over-expression of mutant ataxin-1 that contains an expanded poly-Q domain results in reduced gene expression levels in a mouse model of spinal-cerebellar-ataxia-type 1 (“SCA 1”). The over-expression of mutant ataxin-7 that contains an expanded poly-Q domain results in cone-rod dystrophy presumably by interacting with a cone-rod homeobox transcription factor (“Crx”). In addition, mutant ataxin-1 can bind polyglutamine-binding-protein-1 (“Pqbp1”) resulting in transcription inhibition. CBP can be sequestered into polyglutamine-rich inclusions containing a mutant androgen receptor associated with spinal-bulbar-muscular atrophies (SBMA). CBP can interact with atrophin-1 (“DRPLA”) and ataxin-3 (“SCA 3”). TAF_{II}130, a co-activator of CREB-dependent transcription, also binds to DRPLA with expanded polyglutamine repeats. Animal hosts that can express ataxin-1, ataxin-2, ataxin-3, ataxin-7, atrophin 1, Ncor1, TBP, CBP, Crx, and TAF_{II}30 within the ocular lens can be used to screen for compounds that can inhibit interactions between these neurodegenerative-disease-related polypeptides of the present invention. M. Flint Beal and Robert J. Ferrante, supra.

In another embodiment, the present methods and compositions can be used to screen compounds that inhibit the activity of transglutaminases for the prevention, management, and/or treatment of various polyglutamine-neurodegenerative diseases. Transglutaminase activity might be involved in the pathogenesis of CAG-repeat diseases. In vitro, the transglutaminase inhibitors, cystamine and monodansyl cadaverine, can inhibit the formation of cellular aggregates of truncated DRPLA proteins that contain an expanded-polyglutamine domain. Animal hosts that can express transglutaminases within the ocular lens can be used to screen for compounds that can inhibit the activity of transglutaminases for the treatment of polyglutamine-neurodegenerative diseases. M. Flint Beal and Robert J. Ferrante, supra; and Huda Y. Zoghbi and Harry T. Orr, Ann. Rev. Neurosci., 23:217-247 (2000) are incorporated by reference.

### C. Targets for the Therapy of Parkinson's Disease (PD) and Related Diseases

In one embodiment, the present methods and compositions can be used to screen compounds that can inhibit self-aggregations mediated by wildtype α-synucleins, self-aggregations mediated by mutant α-synuclein variants, aggregations formed by interactions between wildtype and mutant α-synuclein variants for the prevention, management, and/or treatment of Parkinson's disease and related diseases. Related diseases include dementia with Lewy bodies (“DLB”) and multiple system atrophy (“MSA”) that includes olivopontocerebellar atrophy, striatonigral degeneration and Shy-Drager syndrome. The over-expression of wildtype α-synuclein and the over-expression of mutant forms of α-synuclein, can result in pathological phenotypes. Based on experiments using knockout mice deficient in α-synuclein, the wildtype α-synuclein appears to regulate the release of dopamine, an essential neurotransmitter. Mutant α-synuclein variants include mammalian variants of α-synuclein, such as mutant human α-synuclein, mutant human α-synuclein fragment, mutant mouse α-synuclein, mutant mouse α-synuclein fragment, mutant rat α-synuclein, mutant rat α-synuclein fragment, and others. Mutant human α-synuclein variants include A30P α-synuclein, A53T α-synuclein, and other naturally-occurring α-synuclein variants in the human population. Independent over-expression of A30P α-synuclein in which an alanine is substituted by a proline at position 30, and the A53T α-synuclein in which an alanine is substituted by a threonine at position 53 in transfected rats causes selective loss in dopamine-producing neurons of the brain. Mutant mammalian α-synuclein genes can be isolated from naturally-occurring populations, or can be generated in a laboratory by various mutagenesis methods known to persons skilled in the art (Biere et al., U.S. Pat. No. 6,184,351 is incorporated by reference). Mutant variants of α-synuclein include sequences that contain one or more point mutations that result in amino-acid substitutions, sequences containing internal amino-acid deletions, sequences containing internal amino-acid additions, and/or sequences that are truncations of wildtype α-synuclein. Human α-synuclein (also referred to as “NACP”) (NCBI 586067) is designated as SEQ ID NO:2. Michel Goedert, Nature Reviews, Neuroscience, 2:492-501 (2001); Lee et. al, PNAS, 99(13):8968-8973, (2002); Kirik et al., PNAS, 100(5):2884-2889 (2003); and Conway et al., PNAS, 97(2):571-576 (2000) are incorporated by reference.

Polymorphisms in α-synuclein promoter that result in variable levels of α-synuclein expression are thought to contribute to the pathogenesis of PD and related diseases (Papadimitriou et al., Neurology, 52:651-654 (1999) is incorporated by reference). Over-expression of A53T α-synuclein, driven by a prion-related promoter in the brains of transgenic mice induces late-onset motor deficits, paralysis, and death. Although apoptotic cell death did not occur, neuronal intracytoplasmic fibrillary aggregations can be observed in transgenic animals. Over-expression of wildtype α-synuclein in cultured neuroblastoma cells results in fibrillary β-pleated sheet conformations of α-synuclein that leads to apoptosis. Furthermore, over-expression of wildtype α-synuclein is sufficient to cause neural degeneration in transgenic fly, and to cause intracytoplasmic fibrillary aggregations in transgenic mice. Animal hosts that can express wildtype and mutant mammalian α-synuclein variants within the ocular lens can be used to screen for compounds that can inhibit self-aggregations mediated by a wildtype α-synuclein, self-aggregations mediated by mutant α-synuclein, and aggregations formed by interactions between wildtype and mutant α-synuclein variants. Wildtype and mutant mammalian α-synuclein variants may be individually expressed, or both wildtype and mutant mammalian α-synucleins, may be co-expressed in the lens to simulate conditions of early-onset PD in which mixtures of wildtype and mutant α-synuclein, such as A30P α-synuclein or A53T α-synuclein, are present together in PD-diseased brains.

In another embodiment, the present methods and compositions can be used to screen compounds that can inhibit aggregations mediated by wildtype β-synuclein, mutant β-synuclein variant, wildtype γ-synuclein, and mutant γ-synuclein variant, for the prevention, management, and/or treatment of Parkinson's disease and related diseases, including dementia with Lewy bodies (“DLB”), and multiple system atrophy (“MSA”), such as olivopontocerebellar atrophy, striatonigral degeneration and Shy-Drager syndrome. The α-synucleins, β-synucleins, and γ-synucleins share 55-62% identity. The N-terminal portions of the synucleins contain an imperfect 11-amino-acid repeats that bear a consensus sequence KTKEGV. The repeats are followed by a hydrophobic middle region and a negatively-charged carboxy-terminal domain. Both α-synucleins and β-synucleins are found in nerve terminals, and γ-synucleins are present throughout nerve cells. Although β-synucleins and γ-synucleins are not found in Lewy bodies, both are associated with hippocampal axon pathology in PD and dementia with Lewy bodies. A change in γ-synuclein expression is observed in the retina of patients with AD. Julia M. George, Genome Biology, 3(1): 1-6 (2001) is incorporated by reference.

Several heritable forms of PD correlate with gene products encoded by various PD-disease-associated genes, including α-synuclein, parkin, and UCH-L1. Within Lewy bodies, α-synuclein has been shown to bind synphilin-1, parkin, and UCH-L1. In another embodiment, the present methods and compositions can be used to screen compounds that can inhibit interactions between α-synuclein and synphilin-1, between α-synuclein and parkin, and between α-synuclein and UCH-L1, for the prevention, management, and/or treatment of Parkinson's disease and related diseases, including dementia with Lewy bodies (“DLB”), and multiple system atrophy (“MSA”), such as olivopontocerebellar atrophy, striatonigral degeneration and Shy-Drager syndrome. Animal hosts that can co-express synphilin-1, parkin, UCH-L1, β-synuclein, and γ-synuclein, in addition to wildtype or mutant mammalian α-synuclein variants within the ocular lens can be used to screen for compounds that can inhibit α-synuclein-containing aggregations.

In another embodiment, the present methods and compositions can be used to screen compounds that inhibit the activity of transglutaminases for the prevention, management, and/or treatment of PD and related diseases, including dementia with Lewy bodies (“DLB”), and multiple system atrophy (“MSA”), such as olivopontocerebellar atrophy, striatonigral degeneration and Shy-Drager syndrome. In general, transglutaminases catalyze the formation of crosslinkages between glutamine and lysine residues in proteins. In vitro, purified transglutaminases can catalyze crosslinking of α-synuclein that leads to the formation of α-synuclein-containing aggregates. Over-expression of transglutaminases results in the formation of detergent-insoluable, α-synuclein-containing aggregates in cells. Animal hosts that can express transglutaminases within the ocular lens can be used to screen for compounds that can inhibit the activity of transglutaminases for the treatment of PD and PD-related diseases. Junn et. al., PNAS, 100(4):2047-2052 (2003) is incorporated by reference.

### D. Targets for the Therapy of Alzheimer's Disease (AD) and Related Diseases

In one embodiment, the present methods and compositions can be used to identify compounds that can inhibit the self-aggregation of β-amyloid peptides, for the prevention, management, and/or treatment of AD and related diseases. The predisposition to AD has been linked to mutations of several genes, such as amyloid precursor protein (APP), presenilins (“PS-1” and “PS-2”), tau, and apolipoprotein E (APOE), that result in aberrant processing of amyloid precursor protein (APP) into self-aggregating amyloid-β-peptides. For example, suitable mutant presenilin-1 variants include mutants that are disclosed in U.S. Pat. No. 5,986,054. Animal hosts that can co-express neurodegenerative-disease-related polypeptides, such as mutant presenilin 1 (PS-1), mutant presenilin 2 (PS-2), mutant tau, mutant apolipoprotein E (APOE), and wildtype/mutant amyloid precursor protein (APP), within the ocular lens can be used to screen for compounds that can inhibit the formation of self-aggregating β-amyloid peptides that are incorporated as amyloid fibrils.

In another embodiment, the present methods and compositions can be used to identify compounds that can inhibit the activity of proteases that cleave an amyloid precursor protein (APP) into various β-amyloid peptides for the prevention, management, and/or treatment of AD and related diseases. Proteases, such as α-secretases, β-secretases, and γ-secretases, can cleave the APP endogenously, and mutations near cleavage-sites of the APP gene recognized by these secretases promote the generation of deleterious β-amyloid peptides of 39-43 residues. Animal hosts that can co-express neurodegenerative-disease-related polypeptides with protease activity, such as α-secretases, β-secretases, and γ-secretases, in addition to APP, within the ocular lens can be used to screen for compounds that can inhibit the protease activity of these secretases so that production of self-aggregating β-amyloid peptides can be reduced. James Sacchettini and Jeffery Kelly, Nature Reviews, Drug Discovery, 1:267-275 (2002) is incorporated by reference.

Recent investigations suggest that AD and PD pathogenic pathways may overlap. For example, β-amyloid peptides can promote aggregation of α-synuclein in cell-free systems and intraneuronal accumulation of CL-synuclein in cell cultures (Masliah et al., PNAS, 98(21):12245-12250 (2001) is incorporated by reference). In various embodiments, the α-synuclein variants described above may be co-expressed with β-amyloid peptides within the lens of animal hosts to screen compounds for the prevention, management, and/or treatment of Parkinson's disease and related diseases, including dementia with Lewy bodies (“DLB”) and multiple system atrophy (“MSA”), such as olivopontocerebellar atrophy, striatonigral degeneration, Shy-Drager syndrome, AD, and AD-related diseases.

### E. Targets for the Therapy of Amyotrophic Lateral Sclerosis (ALS) and Related Diseases

In one embodiment, the present methods and compositions can be used to screen compounds that can inhibit interactions between variants of mutant SOD1 and other endogenous proteins, for the prevention, management, and/or treatment of amyotrophic lateral sclerosis (ALS) and familial amyotrophic lateral sclerosis (FALS). Wildtype SOD1 encodes an enzyme that catalyzes the dismutation of superoxides, which are free radicals generated as by-products of normal oxidative reactions. Substantial allelic heterogeneity occurs within SOD1 that involves at least 90 different mutations. For example, a single-point mutation within SOD1 accounts for 38% of all FALS, which has been identified as A4V SOD1 in which an alanine is substituted by a valine at codon 4. Transgenic mice carrying the mutant human Gly93Ala SOD1 in which a glycine is substituted by an alanine at position 93 demonstrates ALS phenotype, and a four-fold increase in SOD1 enzymatic activity. Other mutations of SOD1 that can be expressed in the lens of animal hosts of the present invention are disclosed in Brown et al., U.S. Pat. No. 6,723,893, which is incorporated by reference in its entirety. In FALS cases, SOD1 co-localizes with NF-spheroids that contain aggregates of neuronal filaments (NFs), consisting of at least three co-aggregating subunits, low-molecular-weight (“LNF”), medium-molecular-weight (“MNF”), and high-molecular-weight (“HNF”) neuronal filaments. FALS correlates with aggregates of neuronal filaments (NFs), that may be caused by improper protein interactions between mutant SOD1 and other endogenous proteins, such as components of neuronal filaments (NFs). In some ALS cases, the NF-spheroids also contain self-aggregating intermediate filaments (IFs), such as α-internexin and peripherin, within proximal-axonal swellings. Animal hosts that can express mutant SOD1 variants, low-molecular-weight neuronal filament (LNF), medium-molecular-weight neuronal filament (MNF), and high-molecular-weight neuronal filament (HNF), α-internexin, and/or peripherin, within the ocular lens can be used to screen for compounds that can inhibit interactions between SOD1 and components of IFs and NFs for the treatment of ALS and FALS. Tu et al., PNAS, 93:3155-3160 (1996); and Lewis P. Rowland, PNAS, 92:1251-1253 (1995) are incorporated by reference.

### F. Targets for the Therapy of Amyloid Diseases

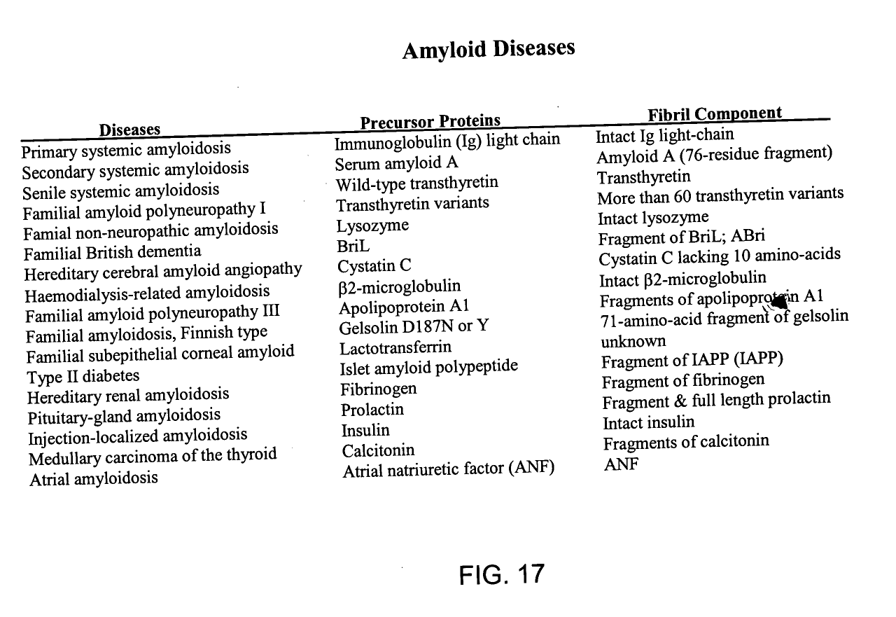

FIG. 17 provides a list of various amyloid diseases that involve self-aggregating polypeptides than form amyloid fibrils. Various amyloid diseases, corresponding precursor proteins, and major components of amyloid fibrils are disclosed. Compounds for treating amyloid diseases, including various amyloid diseases listed FIG. 17, may be screened using the methods and compositions of the present invention.

### EXAMPLES

The following examples are offered by way of illustration and not by way of limitation. In Example 1, methods for constructing transgene expression cassettes containing huntingtin-Exon 1 variants, and methods for constructing non-human transgenic animals expressing EGFP-huntingtin-exon-1 variants are provided. In Example 2, the characterization of the expression of EGFP-huntingtin-exon-1 variants by slit-lamp biomicroscope is provided. In Example 3, microscopic analysis of inclusion bodies containing mutant huntingtin fragments are provided. In Example 4, methods for constructing transgene-expression cassettes containing α-synuclein variants, and methods for constructing non-human transgenic animals expressing EGFP-α-synuclein variants are provided. In Example 5, the characterization of the expression of EGFP-α-synuclein variants by slit-lamp biomicroscope is provided.

### Example 1

### Construction of Transgenic Mice Expressing Huntingtin-Exon-1 Variants within the Ocular Lens

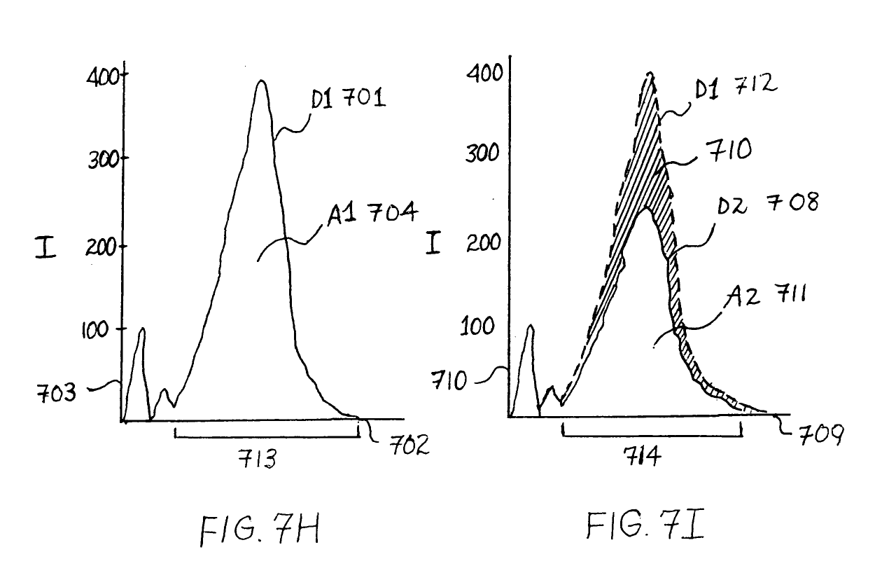

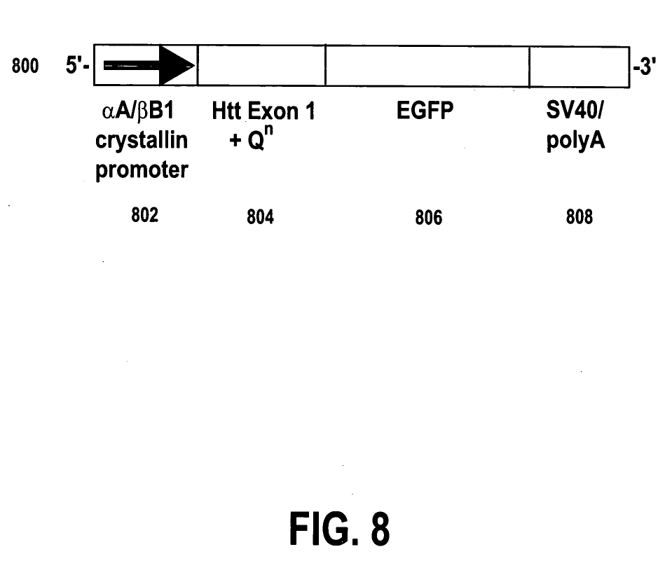

Transgenic mice that can express mutant huntingtin fragments in the ocular lens can be employed for screening compounds to identify active compounds for the prevention, management, and/or treatment of Huntington's disease and related diseases. FIG. 8 is a schematic of an exemplary expression cassette for expressing a gene of interest within the ocular lens of an animal host. In FIG. 8, various forms of EGFP-tagged, huntingtin-exon-1 fragments containing variable lengths of poly-Q-repeat domains can be produced from an expression construct 800 that includes a crystallin promoter 802 positioned upstream and operably-linked to a huntingtin fragment containing an expanded-CAG repeat, such as “Htt Exon1+Qn” 804, which is positioned upstream and operably-linked to an EGFP-encoding sequence 808. Optionally, a 3′ untranslated sequence, such as a “SV40-polyA” sequence 810 or an equivalent sequence may be included at the 3′end of the construct. For generating non-human transgenic mice described below, various transgene-expression cassettes containing fragments of different lengths of expanded-CAG repeats (“HtEx1-47Q,” “HtEx1-72Q,” and “HtEx1-103Q”) are produced. A non-expanded huntingtin fragment (“HtEx1-25Q”) is constructed as a negative control. Each huntingtin fragment is independently and operably-linked to either a murine αA-crystallin promoter, or a chicken βB1-crystallin promoter. Each huntingtin-exon-1 fragment is fused to an EGFP-encoding sequence so that the resulting chimeric protein has the excitation and emission spectra of EGFP.

To construct Huntingtin-EGFP reporter cassettes, huntingtin fragments are removed from a parent plasmid by Bsp120I-mediated restriction enzyme digestion. Various huntingtin fragments and pre-amplified EGFP-encoding sequences are ligated into appropriate cloning sites of vectors containing lens-specific promoters, such as a chicken βB1-crystallin promoter and a mouse αA-crystallin promoter (Taube J. R. et al., Transgenic Research, 11(4):397-410 (2002)). All constructs are confirmed by direct sequencing. Transgene-expression cassettes containing a lens-specific promoter, a huntingtin-exon-1, and an EGFP-encoding sequence are removed from the vectors using AatII and NgoM IV for constructing a αA-crystallin-promoter-regulated cassette, and using EcoRI and PvuI for constructing a βB1-crystallin-promoter-regulated cassette. Each transgene-expression cassettes are purified by agarose-gel electrophoresis and extracted from a gel using a QIAquick kit (Qiagen). DNA is subjected to isopropanol precipitation, pelleted by microfugation, and resuspended in 10 mM Tris, pH 8.0. Products are then subjected to dialysis against 10 mM Tris/0.1 mM EDTA (pH 8.0) prepared from embryo-tested water (Sigma) on a 0.1 μm VCWP filter (Millipore) for 2 hr.

The purified transgenes are used for pronuclear microinjection into mouse embryos of strain B6C3, 75% C57BL/6 and 25% C3H. Founder animals are identified by amplification of DNA from a tail biopsy with primers directed against EGFP by using Taq polymerase (Invitrogen). Positive animals are mated to strain C57BL/6, and the resulting offspring are identified by DNA amplification from tail biopsy. Eight lines of transgenic mice are generated by employing conventional methods. After the characterization of founders, one mouse line can be chosen to screen for inhibitors of poly-Q-mediated aggregation in the lens of transgenic animal hosts.

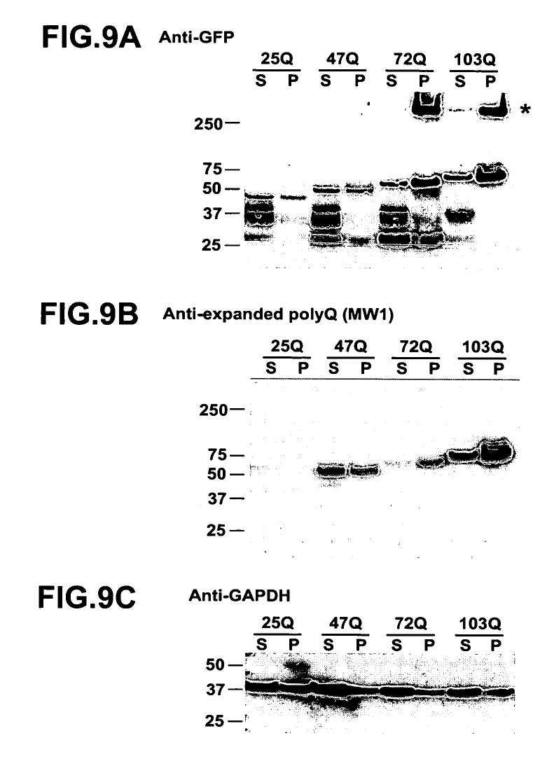

FIG. 9 is an immunoblot analysis of lysates prepared from lens tissues of transgenic animals. Soluable (“S”) and insoluable (“P”) fractions of control lysate (“25Q”) and lysates of transgenic mice that contain various huntingtin transgenes (“47Q,” “72Q,” and “103Q”) are resolved by gel electrophoresis, and the resolved proteins are transferred to nylon blots that are incubated with various monoclonal antibodies, such as an “anti-GFP” antibody as shown in FIG. 9A, an “anti-expanded poly Q” antibody as shown in FIG. 9B, and an “anti-GAPDH” antibody as shown in FIG. 9C. FIG. 9A shows that each huntingtin variant is detectable in both S and P fractions, and that sufficient quantities of the expressed transgene products contain the EGFP epitope. In FIG. 9B, an antibody that specifically recognizes only the expanded poly-Q domain is able to detect the expressed huntingtin transgene variants, but cannot detect the non-expanded control fragment, such as the “25Q.” FIG. 9C shows that comparable amount of control and variant lysates have been analyzed.

### Example 2

### Characterization of the Expression of EGFP-Huntingtin-Exon-1 Fragments within the Ocular Lens of Animal Hosts by Slit-Lamp Biomicroscopy

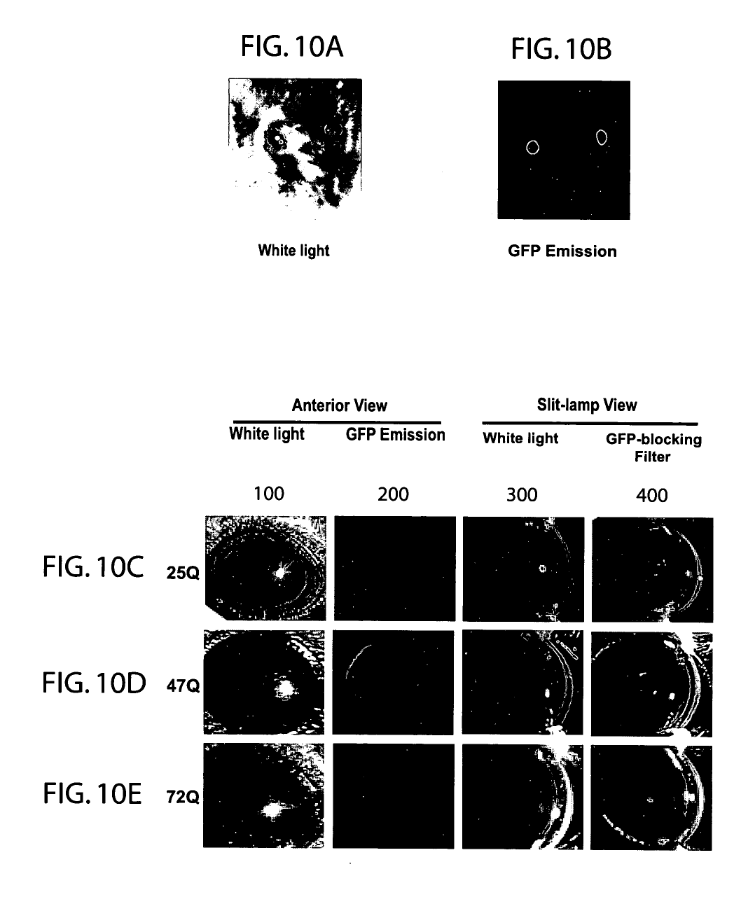

The transgenic animals described in Example 1 are evaluated to quantify poly-Q-mediated-aggregation within the ocular lens of an animal host. In three of the four founder lines, robust lens-specific, or lenticular, expression is observed by employing a slit-lamp biomicroscope that permits the viewing of an optical section of the lens and the digital imaging of GFP fluorescence that arise from the lens of each animal host. FIG. 10 is a digital image of the lens of transgenic founder mice expressing huntingtin-exon-1 variants that contain extended poly-glutamine (Q) domains. FIG. 10A is a digital image of an anterior view of a hypothetical animal host illuminated with white light. FIG. 10B is a digital image of an anterior view of a hypothetical animal host that records the filtered light emitted by GFP-tagged proteins within the ocular lens, under conditions of GFP-excitation and GFP-emission. FIG. 10C shows digital images of the lens of a transgenic mouse expressing a huntingtin-exon-1 fragment containing 25Qs encoded by an exon 1 fragment with a non-expanded-CAG repeat. FIG. 10D shows digital images of the lens of a transgenic mouse expressing a huntingtin-exon-1 fragment containing 47Qs encoded by an exon 1 fragment with an expanded-CAG repeat. FIG. 10E shows digital image of the lens of a transgenic mouse expressing a huntingtin-exon-1 fragment containing 72Qs encoded by an exon 1 fragment with an expanded-CAG repeat. Panel 100 shows an anterior view under white-light excitation. Panel 200 shows an anterior view under conditions of GFP-excitation and GFP-emission. Panel 300 shows a slit-lamp view of the lens under white-light excitation. Panel 400 shows a slit-lamp view of the lens under conditions of GFP-excitation and GFP-exclusion.

For panels 200, in FIGS. 10C-E, a substantial difference in GFP-fluorescence images is noted between the control fragment (“25Q”) labeled with GFP, and the fragments containing an expanded poly-Q domain (“47Q” and “72Q”) labeled with GFP. With respect to the non-expanded huntingtin fragment, HtEx1-25Q, a uniform distribution pattern is observed throughout the cytoplasm of lens cells in 3 lines tested, demonstrating a pattern similar to that observed in other established mammalian cell lines. The control mice that have been monitored at different intervals during development did not develop cataracts in the lens up to 5 months of age. Uniform and diffuse distribution of GFP-tagged protein would suggest a non-aggregated state. Intense GFP fluorescence emerging from fragments that contain expanded-CAG repeats (“47Q” and “72Q”) is detected within the lens cortex. Modest opacities in the lens nucleus (interior sub-region) are detected within 2 months from birth. Comparable distribution patterns are observed by employing either promoters, a mouse αA1-cystallin promoter and a chicken βB1-crystallin promoter.

### Example 3

### Microscopic Analysis of Inclusion Bodies Containing Mutant Huntingtin Fragments

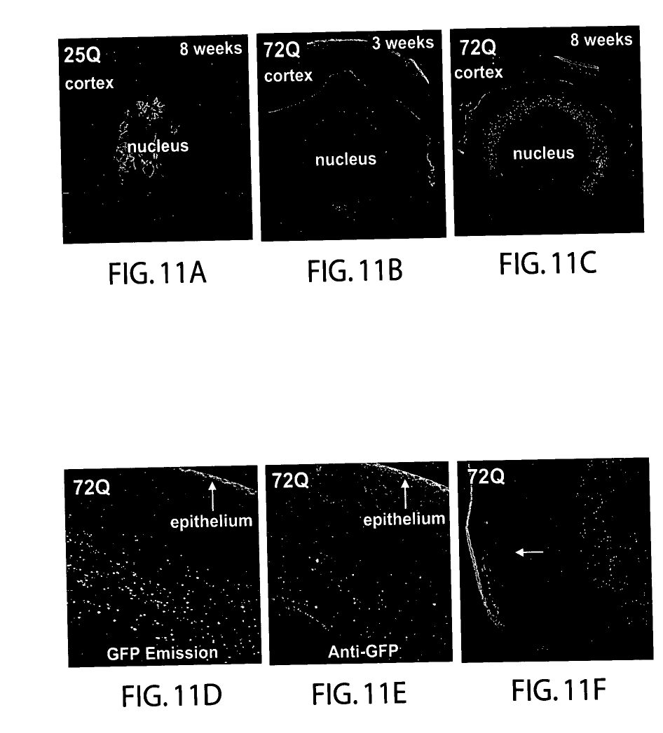

FIG. 11A is a microscopic view of lens that expresses a mutant huntingtin fragment containing a non-extended poly-Q domain “25Q,” removed from a transgenic mouse at 8 weeks of age at 10× magnification. In FIGS. 11A-11C, and 11F, GFP florescence appears in green, and the nuclei stained with propidium iodide stains in red. FIG. 11B illustrates an exemplary lens section removed from a transgenic mouse that shows a thin halo (green) of inclusion bodies at 3 weeks. FIG. 11C illustrates an exemplary lens section removed from a transgenic mouse expressing a mutant huntingtin “72Q” fragment at 8 weeks that shows a ten-fold increase in the number of inclusion bodies within the halo region (green) during this time period. FIG. 11D illustrates an exemplary lens section of lens removed from a transgenic mouse expressing a mutant huntingtin “72Q” fragment examined for GFP fluorescence at 20× magnification. In FIG. 11D, inclusion bodies and aggregates containing GFP-tagged huntingtin fragments appear as white speckles. FIG. 11E illustrates an exemplary lens section of lens removed from a transgenic mouse expressing a mutant huntingtin “72Q” fragment, incubated with an anti-GFP antibody. In FIG. 11E, inclusion bodies and aggregates containing GFP-tagged huntingtin fragments appear as white speckles. Note that the average diameter of inclusion bodies increases in size for cells that are more centrally-located in the lens (within the lens nucleus sub-region), and that only a sub-set of inclusion bodies react with an anti-GFP antibody. FIG. 11F illustrates an exemplary lens section of lens removed from a transgenic mouse expressing a mutant huntingtin “72Q” fragment that is examined for GFP fluorescence. In FIG. 11F, inclusion bodies and aggregates containing GFP-tagged huntingtin fragments that appear as green speckles are observed in the lens cortex that is primarily composed of anucleated cells. In contrast, nucleated lens cells, such as epithelial cells located in the anterior epithelium, appear in red when stained with propidium.

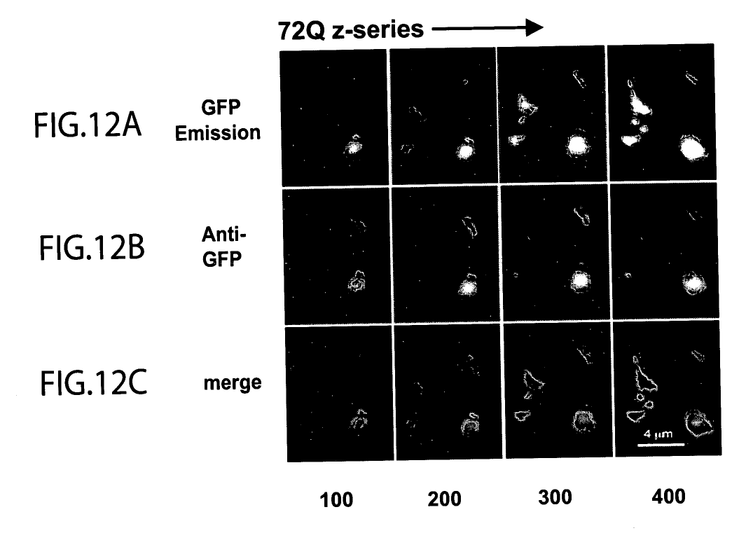

FIGS. 12A-C illustrate Z-series projections at 60× magnification scanned through several inclusion bodies within a lens cell, which are isolated from a transgenic mouse expressing a mutant huntingtin “72Q” fragment. Panels positioned from left to right, from panels 100 to panels 400, represent different cross-sections of aggregates, which are scanned at increasing depths. FIG. 12A shows GFP fluorescence emitted from inclusion bodies containing GFP-tagged huntingtin fragments. FIG. 12B shows inclusion bodies containing GFP-tagged huntingtin fragments labeled with an anti-GFP antibody. FIG. 12C shows a composite or merged image of panels in FIG. 12A with panels in FIG. 12B that are vertically-positioned. Note that the anti-GFP antibody reacts with only a subset of inclusion bodies.

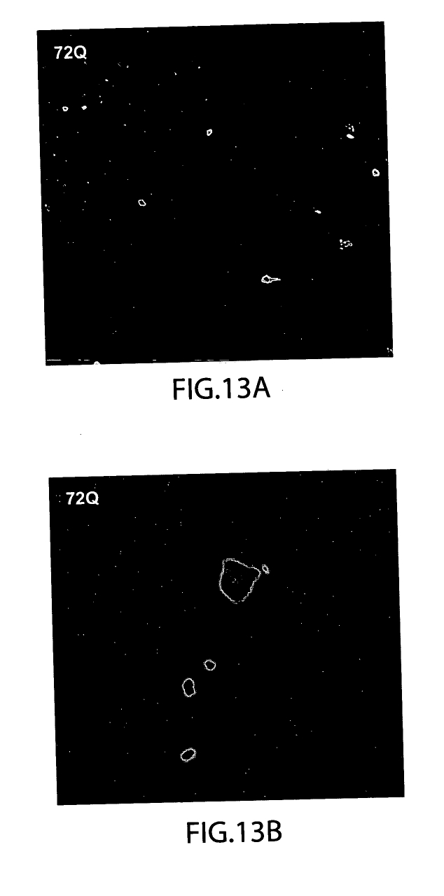

FIGS. 13A-B illustrate a cross-section of a lens fiber cell showing inclusion bodies of varying sizes (1-5 μm) formed in the lens. GFP fluorescence appears as green, and lens-fiber-cell membranes labeled with wheat-germ agglutinin appear in red.

### Example 4

### Construction of Transgenic Mice Expressing α-Synuclein Variants within the Ocular Lens

Transgenic mice that can express α-synuclein in the ocular lens can be employed for screening compounds to identify compounds for the prevention, management, and/or treatment of Parkinson's disease and related diseases. An expression cassette can be constructed that includes a crystallin promoter, which is positioned upstream and operably-linked to an α-synuclein variant, which is positioned upstream and operably-linked to an EGFP-encoding sequence. Optionally, a 3′ untranslated sequence, such as a “SV40-polyA” sequence, or an equivalent sequence, may be included at the 3′end of the construct. Variants of α-synuclein that can be expressed in the lens of an animal host include wildtype α-synuclein and two well-characterized point mutations of α-synuclein, in particular, the A53T α-synuclein, resulting from a substitution of alanine for threonine at position 53, and the A30P α-synuclein, resulting from a substitution of alanine for serine at position 30. Each α-synuclein fragment is independently and operably-linked to either a murine αA-crystallin promoter, or a chicken βB1-crystallin promoter. Each α-synuclein variant is fused to an EGFP-encoding sequence so that the resulting chimeric protein has the excitation and emission spectra of EGFP.

To construct α-synuclein variant reporter cassettes, α-synuclein variants are removed from a parent plasmid by SpeI and XhoI-mediated restriction enzyme digestion. Various α-synuclein variants and pre-amplified EGFP-encoding sequences are ligated into appropriate cloning sites of vectors containing lens-specific promoters, such as a chicken βB1-crystallin promoter and a mouse αA-crystallin promoter (Taube J. R. et al., Transgenic Research, 11(4):397-410 (2002)). All constructs are confirmed by direct sequencing. Transgene-expression cassettes containing a lens-specific promoter, such as a βB1-crystallin promoter, a α-synuclein variant, and an EGFP-encoding sequence are removed from the vectors using KpnI and BglI. Each transgene-expression cassettes are purified by agarose-gel electrophoresis and extracted from a gel using a QIAquick kit (Qiagen). DNA is subjected to isopropanol precipitation, pelleted by microfugation, and resuspended in 10 mM Tris, pH 8.0. Products are then subjected to dialysis against 10 mM Tris/0.1 mM EDTA (pH 8.0) prepared from embryo-tested water (Sigma) on a 0.1 μm VCWP filter (Millipore) for 2 hr. After the characterization of founders, one mouse line can be chosen to screen for inhibitors of α-synuclein-mediated aggregation in the lens of an animal host.

### Example 5

### Characterization of the Expression of EGFP-α-Synuclein Variants within the Ocular Lens of Animal Hosts by Slit-Lamp Biomicroscopy

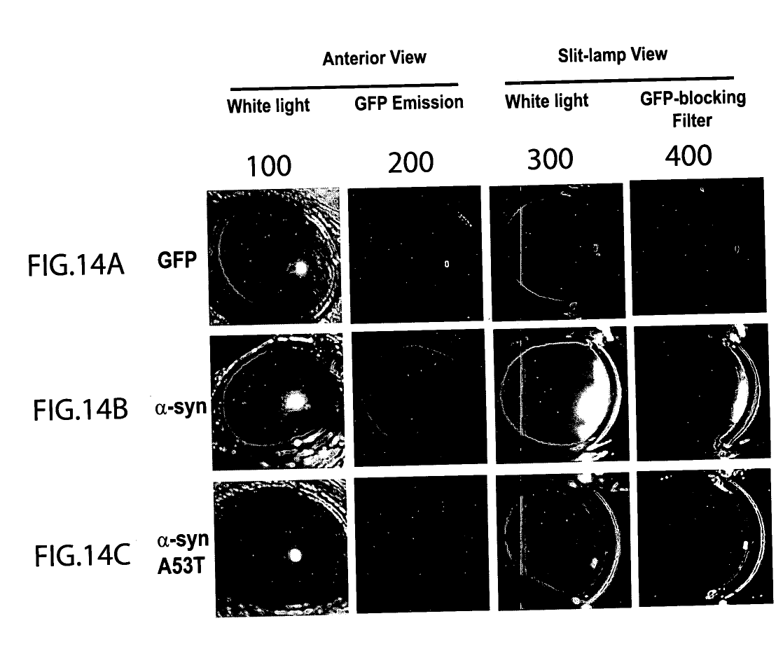

FIGS. 14A-C are digital images of the lens of transgenic mice that express α-synuclein variants. FIG. 14A shows digital images of the lens of a GFP-expressing transgenic mouse. FIG. 14B shows digital images of the lens of a transgenic mouse expressing a wildtype human α-synuclein. FIG. 14C shows digital images of the lens of a transgenic mouse expressing a mutant human α-synuclein, “α-syn-A53T.” Panel 100 shows an anterior view under white-light excitation. Panel 200 shows an anterior view under conditions of GFP-excitation and GFP-emission. Panel 300 shows a slit-lamp view of the lens under white-light excitation. Panel 400 shows a slit-lamp view of the lens under conditions of GFP-excitation and GFP-exclusion.

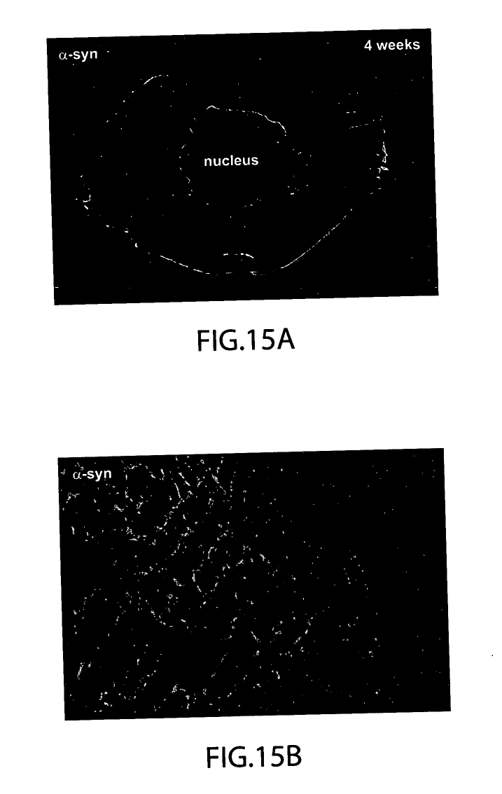

FIGS. 15A-B show digital images of microscopic sections of lens removed from a transgenic mouse expressing α-synuclean at 4 weeks of age, at 10× and 60× magnification, respectively. GFP fluorescence appears in green, and lens-fiber-cell membranes labeled with wheat-germ agglutinin appear in red.

Although the present invention has been described in terms of particular embodiments, it is not intended that the invention be limited to these embodiments. Modifications within the spirit of the invention will be apparent to those skilled in the art. The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and practical applications, and thereby enable others skilled in the art to best utilize the invention and various embodiments with various modifications as are suited to particular uses contemplated. It is intended that the scope of the invention be defined by the following claims and their equivalents:

1. A method for screening compounds to identify active compounds, the method comprising:
determining an amount of aggregates within an ocular lens of a first animal host that has been genetically engineered to express one or more neurodegenerative-disease-related polypeptides encoded by a gene of interest, within an ocular lens of the host animal, and that has not been exposed to a test compound;
determining an amount of aggregates within an ocular lens of a second animal host that is genetically identical to the first animal host, and that has been exposed to the test compound;
comparing the determined amount of aggregates within the lens of the second animal host relative to the determined amount of aggregates within the lens of the first animal host; and
characterizing the test compound as active when the determined amount of aggregates observed in the lens of the second animal host is less than the determined amount of aggregates observed in the lens of the first animal host.

2. The method of claim 1 wherein the active compound is used for the prevention, management, and/or treatment of a neurodegenerative disease.

3. The method of claim 1 wherein the active compound is used for determining the bioavailability of the test compound within a brain of the second animal host.

4. The method of claim 1 wherein the first animal host is a control for determining a reference measurement.

5. The method of claim 1
wherein the first animal host is the same animal as the second animal host; and
wherein determining the amount of aggregates further includes evaluating the first animal host prior to evaluating the second animal host.

6. The method of claim 1 wherein the determining the amount of aggregates in the lens of the first and the second animal hosts further includes measuring the intensity of light photons scattered from the aggregates containing neurodegenerative-diseases-related polypeptides.

7. The method of claim 1 wherein the determining the amount of aggregates in the lens of the first and the second animal hosts further includes measuring the intensity of fluorescence emission from the neurodegenerative-diseases-related polypeptides labeled with a fluorophore.

8. The methods of claim 1 wherein the compound decreases the amount of aggregates detected in the first animal host by at least about 10%.

9. The method of claim 1 wherein the compound is systemically administered to the first animal host.

10. The method of claim 1 wherein the neurodegenerative disease includes at least one of: Huntington's disease (HD), CAG-repeat expansion disease, dentatorubral-pallidoluysian atrophy (DRPLA), spinal and bulbar muscular atrophy (SBMA), spinocerebellar ataxia type-1 (SCA 1), spinocerebellar ataxia type-2 (SCA 2), spinocerebellar ataxia type-3 (SCA 3), alpha 1a-voltage dependent calcium channel, spinocerebellar ataxia type-6 (SCA 6), spinocerebellar ataxia type-7 (SCA 7), Machado-Joseph disease (MJD), Parkinson's disease (PD), dimensia of Lewy bodies (“DLB”), multiple system atrophy (“MSA”), olivopontocerebellar atrophy, striatonigral degeneration, Shy-Drager syndrome, amyotrophic lateral sclerosis (ALS), familial amyotrophic lateral sclerosis (FALS), Lou Gehrig's disease, Charcot disease, motor neuron disease, Creutzeldt-Jakob disease, progressive supranucler palsy, Gerstmann-Straussler-Scheinker syndrome, fetal familial insomnia, Alzheimer's Disease (AD), and amyloid diseases.

11. The method of claim 1 wherein the neurodegenerative-disease-related polypeptide is a self-aggregating protein, or a self-aggregating fragment thereof, that is one of: huntingtin, atrophin-1, androgen receptor, ataxin-1, ataxin-2, ataxin-3, CACNA1A, ataxin-7, α-synuclein, amyloid precursor protein (APP), tau, β-amyloid peptide, low-molecular-weight neuronal filament (LNF), medium-molecular-weight neuronal filament (MNF), high-molecular-weight neuronal filament (HNF), α-internexin, and peripherin.

12. The method of claim 1 wherein the neurodegenerative-disease-related polypeptide is at least one of: huntingtin, N-Cor, mSin3a, CBP (c-AMP-responsive element-binding protein), α-adaptin, α1-antichymotrypsin, α-synuclein, β-synuclein, γ-synuclein, synphilin-1, parkin, UCH-L1, tau, caspase-1, caspase-2, caspase-3, caspase-6, caspase-8, calpain, aspartyl protease, neuronal cytochrome c, SP1, histone deacetylases (HDAC), transglutaminases, polyglutamine-binding-protein-1 (PQBP1), SOD1, apolipoprotein E (APOE), TAFI_{II}30, Crx, hap-1, GAPDH, PSD95, CA150, TBP (TATA-binding protein), PS-1, PS-2, α-secretase, β-secretase, and γ-secretase.

13. The method of claim 1 wherein the gene of interest is transcriptionally regulated by a lens-specific promoter.

14. The method of claim 1 wherein the first and the second animal hosts express the neurodegenerative-disease-related polypeptide in a central nervous tissue of the first and second animal hosts.

15. The method of claim 1 wherein the animal host is a non-human transgenic animal.

16. The method of claim 1 wherein the gene of interest contains an expanded-CAG repeat, and encodes a mammalian mutant polypeptide.

17. The method of claim 16 wherein the mammalian mutant polypeptide includes at least one of: huntingtin, atrophin-1, androgen receptor, ataxin-1, ataxin-2, ataxin-3, CACNA1A, and ataxin-7.

18. The method of claim 1 wherein the gene of interest encodes a mammalian variant of α-synuclein that includes at least one of:
a mutant human α-synuclein polypeptide that contains one or more amino-acid substitutions with respect to a wildtype human α-synuclein, wherein the mutant polypeptide self-aggregates, or aggregates with other biomolecules;
a mutant mouse α-synuclein polypeptide that contains one or more amino-acid substitutions with respect to a wildtype mouse α-synuclein, wherein the mutant polypeptide self-aggregates, or aggregates with other biomolecules;
a mutant rat α-synuclein polypeptide that contains one or more amino-acid substitutions with respect to a wildtype rat α-synuclein, wherein the mutant polypeptide self-aggregates, or aggregates with other biomolecules;
a mutant A30P human α-synuclein polypeptide that contains a substitution of a proline at position 30 for an alanine, with respect to SEQ ID NO 2; and
a mutant A53T human α-synuclein polypeptide that contains a substitution of a threonine at position 53 for an alanine, with respect to SEQ ID NO 2.

19. An animal host for screening compounds to identify active compounds for the prevention, management, or treatment of a neurodegenerative disease, the animal host comprising:
a gene of interest that encodes a neurodegenerative-disease-related polypeptide; and
a promoter that is operably-linked to the gene of interest, and that activates the transcription of the gene of interest in an ocular lens of an animal host.

20. The animal host of claim 19 is a non-human transgenic animal.

21. The animal host of claim 19 wherein the promoter includes a lens-specific promoter.

22. The animal host of claim 19 wherein the gene of interest encodes a fusion protein.

23. The animal host of claim 22 wherein the fusion protein includes a fluorescent polypeptide.

24. The animal host of claim 19 wherein the neurodegenerafive-disease-related polypeptide is a self-aggregating protein, or a self-aggregating fragment thereof, that is one of: huntingtin, atrophin-1, androgen receptor, ataxin-1, ataxin-2, ataxin-3, CACNA1A, ataxin-7, α-synuclein, amyloid precursor protein (APP), tau, β-amyloid peptide, low-molecular-weight neuronal filament (LNF), medium-molecular-weight neuronal filament (MNF), high-molecular-weight neuronal filament (HNF), α-internexin, and peripherin.

25. The method of claim 19 wherein the neurodegenerative-disease-related polypeptide is at least one of: huntingtin, N-Cor, mSin3a, CBP (c-AMP-responsive element-binding protein), α-adaptin, α1-antichymotrypsin, α-synuclein, β-synuclein, γ-synuclein, synphilin-1, parkin, UCH-L1, tau, caspase-1, caspase-2, caspase-3, caspase-6, caspase-8, calpain, aspartyl protease, neuronal cytochrome c, SP1, histone deacetylases (HDAC), transglutaminases, polyglutamine-binding-protein-1 (PQBP1), SOD1, apolipoprotein E (APOE), TAFI_{II}30, Crx, hap-1, GAPDH, PSD95, CA150, TBP (TATA-binding protein), PS-1, PS-2, α-secretase, β-secretase, and γ-secretase.

26. The animal host of claim 19 wherein the neurodegenerative disease includes at least one of: Huntington's disease (HD), CAG-repeat expansion disease, dentatorubral-pallidoluysian atrophy (DRPLA), spinal and bulbar muscular atrophy (SBMA), spinocerebellar ataxia type-1 (SCA 1), spinocerebellar ataxia type-2 (SCA 2), spinocerebellar ataxia type-3 (SCA 3), alpha 1a-voltage dependent calcium channel, spinocerebellar ataxia type-6 (SCA 6), spinocerebellar ataxia type-7 (SCA 7), Machado-Joseph disease (MJD), Parkinson's disease (PD), dimensia of Lewy bodies (“DLB”), multiple system atrophy. (“MSA”), olivopontocerebellar atrophy, striatonigral degeneration, Shy-Drager syndrome, amyotrophic lateral sclerosis (ALS), familial amyotrophic lateral sclerosis (FALS), Lou Gehrig's disease, Charcot disease, motor neuron disease, Creutzeldt-Jakob disease, progressive supranucler palsy, Gerstmann-Straussler-Scheinker syndrome, fetal familial insomnia, Alzheimer's Disease (AD), and amyloid diseases.

27. The animal host of claim 19 wherein the gene of interest contains an expanded-CAG repeat, and encodes a mammalian mutant polypeptide.

28. The method of claim 19 wherein the mammalian mutant polypeptide includes at least one of: huntingtin, atrophin-1, androgen receptor, ataxin-1, ataxin-2, ataxin-3, CACNA1A, and ataxin-7.

29. The animal host of claim 19 wherein the transgene of interest encodes a mammalian variant of α-synuclein that includes at least one of:
a mutant human α-synuclein polypeptide that contains one or more amino-acid substitutions with respect to a wildtype human α-synuclein, wherein the mutant polypeptide self-aggregates, or aggregates with other biomolecules;
a mutant mouse α-synuclein polypeptide that contains one or more amino-acid substitutions with respect to a wildtype mouse α-synuclein, wherein the mutant polypeptide self-aggregates, or aggregates with other biomolecules;
a mutant rat α-synuclein polypeptide that contains one or more amino-acid substitutions with respect to a wildtype rat α-synuclein, wherein the mutant polypeptide self-aggregates, or aggregates with other biomolecules;
a mutant A30P human α-synuclein polypeptide that contains a substitution of a proline at position 30 for an alanine, with respect to SEQ ID NO 2; and
a mutant A53T human α-synuclein polypeptide that contains a substitution of a threonine at position 53 for an alanine, with respect to SEQ ID NO 2.

30. An animal host for screening compounds to identify active compounds for the prevention, management, and/or treatment of a neurodegenerative disease, the animal host comprising:
a first gene of interest that encodes a neurodegenerative-disease-related polypeptide;
a promoter that is operably-linked to the first gene of interest, and that activates the transcription of the first gene of interest within an ocular lens of an animal host;
a second gene of interest that encodes a neurodegenerative-disease-related polypeptide; and
a promoter that is operably-linked to the second gene of interest, and that activates the transcription of the second gene of interest within a central nervous tissue of the animal host.

31. The animal host of claim 30 wherein the first gene of interest and the second gene of interest are the same.

32. The animal host of claim 30 wherein the central nervous tissue is a brain of the animal host.

33. An expression vector or an expression cassette, the expression vector or the expression cassette comprising:
a gene of interest that encodes a neurodegenerative-disease-related polypeptide; and
a promoter that is operably-linked to the gene of interest, and that activates the transcription of the gene of interest in an ocular lens of an animal host.
